(19) United States
(12) Patent Application Publication
(10) Pub. No.: US 2025/0152633 A1
(43) Pub. Date: May 15, 2025

(54) CELLS HAVING IMPROVED PROLIFERATIVE CAPACITY AND REDUCED CELLULAR SENESCENCE

(71) Applicant: BOARD OF REGENTS, THE UNIVERSITY OF TEXAS SYSTEM, Austin, TX (US)

(72) Inventors: Aaron BAKER, Austin, TX (US); Miles MASSIDDA, Austin, TX (US)

(73) Assignee: BOARD OF REGENTS, THE UNIVERSITY OF TEXAS SYSTEM, Austin, TX (US)

(21) Appl. No.: 18/884,506

(22) Filed: Sep. 13, 2024

Related U.S. Application Data
(60) Provisional application No. 63/582,416, filed on Sep. 13, 2023.

(51) Int. Cl. A61K 35/28; C12N 5/0775; C12N 13/00

(52) U.S. Cl. CPC A61K 35/28; C12N 5/0663; C12N 13/00; C12N 2500/32; C12N 2501/727; C12N 2501/999

(57) ABSTRACT

Provided herein are methods for mechanically conditioning cells to improve their proliferative function and multipotency, as well as protect them from advancing senescence. In the methods, brachial loading alone is first used to improve the efficiency of culture expansion. Once a sufficient population of healthy cells are generated, pharmacological agents are added as a cotreatment to drive differentiation into a desired phenotype. The conditioned cells may then be used to prepare compositions for administration to a patient to treat a disease.

### REFERENCE TO RELATED APPLICATIONS

The present application claims the priority benefit of U.S. provisional application No. 63/582,416, filed Sep. 13, 2023, the entire contents of which are incorporated herein by reference.

### STATEMENT OF FEDERALLY SPONSORED RESEARCH

This invention was made with government support under Grant no. R01 HL141761 awarded by the National Institutes of Health. The government has certain rights in the invention.

### BACKGROUND

### 1. Field

The present invention relates generally to the fields of cell biology and cellular therapeutics. More particularly, it concerns methods of rejuvenating senescent cells as well as cells made by those methods and their use in therapy.

### 2. Description of Related Art

Bone marrow-derived mesenchymal stem cells (MSCs) are promising candidates for cell-based therapies for many diseases. MSCs are readily available in adult patients, do not require genetic modification for multipotency, and secrete bioactive factors for immunomodulation and angiogenesis (Yong et al., 2018; Clayton et al., 2017). However, current MSC therapies have several key limitations, including suboptimal regenerative phenotype (Murphy et al., 2013), limited potential for in vitro expansion and poor efficacy in aged or diabetic patients (Li et al., 2016). A major key limiter of the regenerative function of MSCs is cellular senescence, which occurs more rapidly in MSCs from aged patients and during cell culture expansion (Zhou et al., 2020). Senescence limits the efficacy of autologous MSC therapies in patients who are elderly (Kuang et al., 2015; Lin et al., 2019) or have co-morbidities like diabetes (Loomans et al., 2004; Vecellio et al., 2014).

Studies have attempted to use MSCs derived from young, allogenic donors to bypass the hurdles poised by senescence in autologous therapies, but the long-term ability of allogenic MSCs to enhance tissue regeneration is limited by an immune response brought on by the transition of the cells from an immunoprivileged to immunogenic state following implantation in the patient (Huang et al., 2010; Isakova et al., 2014). During culture expansion, MSCs experience a loss of differentiation potential and self-renewal capacity (Bonab et al., 2006), shortening of telomeres and chromosomal instability (Drela et al., 2019), metabolic dysfunction (Yuang et al., 2020), and loss of immunomodulatory functions (Fafián-Labora et al., 2019). Thus, senescence limits the usefulness of MSCs from older donors and those with poor health and reduces the in vitro expansion of MSCs, limiting the number of cells that can be generated for therapeutic use.

While the functional characteristics of MSC senescence are well described (Zhou et al., 2020; Turinetto et al., 2016; Chen et al., 2019), the molecular mechanisms that contribute to the development of senescence are less well defined and often multifactorial. Senescent MSCs have limited proliferation capability (Bruder et al., 1997), diminished multipotency (Siegel et al., 2013), and reduced regenerative properties (Yuan et al., 2020). Reactive oxygen species (ROS) byproducts are produced as MSCs age and replicate, causing stress-induced senescence, in which defective antioxidant mechanisms allow for ROS to build up in the mitochondria and lead to metabolic dysfunction and loss of proliferative capability (Kornienko et al., 2019; Denu & Hematti, 2016; Vono et al., 2017). Oxidative stress and other stress stimuli can cause DNA damage, and dysregulated DNA damage repair pathways in aged MSCs can lead to apoptosis or the induction of senescence (Kosar et al., 2011; Banimohamad-Shotorbani et al., 2020). The secretion of soluble factors is also a major aspect in MSC tissue repair and renewal of vasculature (Ahmadi & Rezaie, 2021). As cells become senescent, they adopt a state known as a senescence-associated secretory phenotype (SASP) and cause widespread ECM remodeling and tissue damage via the activation of inflammatory cascades (Lunyak et al., 2017).

Studies have shown that targeting these intrinsic mechanisms of senescence may reverse its progression, providing hope for autologous MSC therapies (Yuang et al., 2020; Stolzing et al., 2008; Meng et al., 2020; Khanh et al., 2020). However, improved methods for reducing cellular senescence in MSC are needed.

### SUMMARY

Provided herein are biomechanical conditioning treatments for rejuvenating MSCs derived from elderly or diseased patients and MSCs undergoing extensive expansion in cell culture. The enhanced properties of the treated cells provide a significant therapeutic advantage over non-treated, senescent MSCs.

Provided herein are methods of preparing therapeutic cell populations, the methods comprising:
(a) adhering a starting population of cells on a flexible or expandable surface;
(b) applying a brachial waveform of mechanical stretch to the starting population of cells, thereby generating a second population of cells;
(c) expanding the second population of cells under static conditions; and
(d) applying a brachial waveform of mechanical stretch to the expanded second population of cells in the presence of at least one pharmacological agent that induces differentiation of the cells, thereby generating the therapeutic population of cells.

The brachial waveform mechanical stretch in step (b) and/or step (d) may be applied to the cells for 1 day, 2 days, 3 days, 4 days, 5 days, 6 days, 7 days, 8 days, 9 days, or 10 days. The brachial waveform mechanical stretch in step (b) and/or step (d) may be applied to the cells for about 1 hour, about 2 hours, about 3 hours, about 4 hours, about 5 hours, about 6 hours, about 7 hours, or about 8 hours per day. In one aspect, the brachial waveform mechanical stretch in step (b) and/or step (d) is applied to the cells for about 4 hours per day for 7 days.

The brachial waveform in step (b) and/or step (d) may have a frequency of 0.01 Hz-1.00 Hz. The brachial waveform in step (b) and/or step (d) may have a frequency of 0.1 Hz, 0.2 Hz, 0.3 Hz, 0.4 Hz, 0.5 Hz, 0.6 Hz, 0.7 Hz, 0.8 Hz, or 0.9 Hz.

The brachial waveform in step (b) and/or step (d) may have a magnitude of strain of 0.1% to 17.5%. The brachial waveform in step (b) and/or step (d) may have a magnitude of strain of 0.1%, 1%, 2.5%, 5%, 7.5%, 10%, 12.5%, 15%, or 17.5%.

In one aspect, the brachial waveform in step (b) and/or step (d) has a magnitude of 7.5% strain and a frequency of 0.1 Hz.

In some aspects, step (b) of the methods may comprise applying a brachial waveform of mechanical stretch to the starting population of cells for about 4 hours per day for about 7 days; step (c) may comprise expanding the second population of cells under static conditions for at least about 1 week, 2 weeks, 3 weeks, 4 weeks, 5 weeks, 6 weeks, 7 weeks, or 8 weeks; and step (d) may comprise applying a brachial waveform of mechanical stretch to the expanded second population of cells in the presence of at least one pharmacological agent that induces differentiation of the cells for about 4 hours per day for about 7 days.

The at least one pharmacological agent that induces differentiation of the cells may be an agent that inhibits signaling of at least one ErbB family protein. The agent that inhibits signaling of at least one ErbB family protein may inhibit EGFR/ErbB1 signaling, HER2/ErbB2 signaling, HER4/ErbB4 signaling, or EGFR/PKC signaling. The agent that inhibits signaling of at least one ErbB family protein may enhance Smad2/3 and/or Hippo pathway activation. The agent that inhibits signaling of at least one ErbB family protein may be a kinase inhibitor, such as, for example, an EGFR/Erb-2/4 inhibitor or a PKCβII/EGFR inhibitor.

In some aspects, step (b) may comprise applying a brachial waveform of mechanical stretch to the starting population of cells for about 4 hours per day for about 7 days; step (c) may comprise expanding the second population of cells under static conditions for at least about 1 week, 2 weeks, 3 weeks, 4 weeks, 5 weeks, 6 weeks, 7 weeks, or 8 weeks; and step (d) may comprise applying a brachial waveform of mechanical stretch to the expanded second population of cells in the presence of at least one pharmacological agent that inhibits signaling of at least one ErbB family protein for about 4 hours per day for about 7 days.

The starting population of cells may be or may have been obtained from a donor at least 60 years of age. The starting population of cells may be or may have been obtained from a diabetic donor. The starting population of cells may have previously undergone in vitro expansion. The starting population of cells may be senescent.

The cells may be mesenchymal stem cells. The cells may be senescent mesenchymal stem cells. The cells may be T cells. The cells may be CAR T cells. The cells may be a therapeutic cell type or cell type used in organotypic models of disease or physiology such as human progenitor cells, endothelial cells, vascular smooth muscles, fibroblasts, cardiomyocytes, skin cells, liver cells, or epithelial cells.

The methods may reverse the senescence of the starting cell population. The population of cells produced by step (d) may comprise fewer senescent cells as compared to the starting population of cells. The method may prevent senescence in the population of therapeutic cells.

The population of therapeutic cells may have increased multipotency as compared to the starting population of cells. The population of therapeutic cells may have increased multipotency as compared to a cell population generated by culturing the starting population of cells under static conditions. The population of therapeutic cells may have increased proliferative capacity as compared to the starting population of cells. The population of therapeutic cells may have increased proliferative capacity as compared to a cell population generated by culturing the starting population of cells under static conditions. The population of therapeutic cells may have improved regenerative properties as compared to the starting population of cells. The population of therapeutic cells may have improved regenerative properties as compared to a cell population generated by culturing the starting population of cells under static conditions. The population of therapeutic cells may comprise fewer senescent cells as compared to the starting population of cells. The population of therapeutic cells may comprise fewer senescent cells as compared to a cell population generated by culturing the starting population of cells under static conditions.

The second population of cells may have an enhanced proliferative function as compared to the starting population of cells. The second population of cells may have an enhanced proliferative capacity as compared to a cell population generated by culturing the starting population of cells under static conditions. The second population of cells may comprise fewer senescent cells as compared to the starting population of cells. The second population of cells may comprise fewer senescent cells as compared to a cell population generated by culturing the starting population of cells under static conditions. The second population of cells may comprise decreased DNA damage as compared to the starting population of cells. The second population of cells may comprise decreased DNA damage as compared to a cell population generated by culturing the starting population of cells under static conditions.

The methods may further comprise step (e) encapsulating the population of therapeutic cells in a gel delivery vehicle. The gel delivery vehicle may be an alginate-RGD-collagen gel delivery vehicle.

The methods may further comprise step (e) culturing the population of therapeutic cells on a tissue engineering scaffold to generate a tissue-engineered construct.

Provided herein are populations of therapeutic cells produced by the methods described herein. The populations of therapeutic cells produced by the methods described herein may be used in the manufacture of a medicament for the treatment of a disease or an injury in a patient.

Provided herein are compositions comprising the populations of therapeutic cells described herein. The cells may be encapsulated in a gel delivery vehicle. The cells may be encapsulated in an alginate-RGD-collagen gel delivery vehicle. The cells may be comprised in a tissue-engineered construct. The tissue engineering scaffold may be a vascular graft, an arteriovenous graft, an artery graft, a vein graft, or a coronary graft. The compositions may be for use in the treatment of a patient with a disease or an injury.

Provided herein are methods of treating a patient in need thereof comprising administering to the patient a therapeutically effective amount of the compositions comprising the populations of therapeutic cells described herein. The patient may have a cardiovascular disease, may be receiving vascular grafts, may have ischemia, may have a healing wound, may have peripheral ischemia, or may have peripheral vascular disease.

Administering may comprise injecting or implanting the cells into the patient. The composition or the cells may be implanted into the patient at the site of diseased tissue.

The composition or the cells may be implanted into the patient at the site of vascular dysfunction. The patient may have peripheral artery disease or ischemia. The methods may induce therapeutic angiogenesis.

The starting population of cells may be mesenchymal stem cells harvested from the bone marrow or adipose tissue of the patient. The population of therapeutic cells may be autologous to the patient.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1H. Mechanical treatment of MSCs reverses correlation of gene expression profile with senescence related gene sets. MSCs from three young donors were conditioned with mechanical and pharmaceutical treatments and analyzed with RNA sequencing. A transcriptomic analysis was performed to compare the gene expression of treated MSCs with the gene expression patterns of key senescence-related genes in MSCs. Results revealed the reversal of senescence-related gene expression patterns with mechanical and pharmaceutical treatment in gene sets for: (FIG. 1A) Aging senescence-related genes. (FIG. 1B) Oncogenic senescence-related genes. (FIG. 1C) Oxidative stress senescence-related genes. (FIG. 1D) Developmental senescence-related genes. (FIG. 1E) Inflammation mediating genes. Next, a Gene Set Enrichment Analyses was performed to compare the gene enrichment in MSCs from young donors after control or brachial loading treatment using predefined gene sets for: (FIG. 1F) Genes upregulated in high passage MSCs. FDR q-value=0.0011. (FIG. 1G) Genes upregulated in elderly donor derived MSCs. FDR q-value=0.0267. (FIG. 1H) Genes upregulated in senescent cells. FDR q-value=0.0151.

FIGS. 2A-2D. Mechanical and pharmaceutical treatments enhance proliferation of aged MSCs. MSCs derived from three aged donors were treated with brachial loading and E/E inhibitor and assessed for potential rejuvenation of proliferative ability. (FIG. 2A) Quantification of BrDU cell proliferation assay. *p<0.05 vs. static control treatment. (FIG. 2B) Quantification of the cumulative doubling of the treated cells from an 81-year-old female donor during long-term cell culture expansion. *p<0.05 vs. static control treatment. (FIG. 2C) Calculation of the number of cells produced from 100K cells during expansion in the different groups. (FIG. 2D) Western blotting results and quantification for proliferation-related protein expression after treatment of MSCs. *p<0.05 vs. static control treatment.

FIGS. 3A-3D. Mechanical conditioning enhances differentiative capability of aged MSCs. MSCs derived from one aged donor were treated with brachial loading and E/E inhibitor, expanded until P8, then differentiated into adipogenic and osteogenic lineages. (FIG. 3A) Adipogenic staining results and (FIG. 3B) quantification FABP4+ cells. *p<0.05 vs. static control treatment. (FIG. 3C) Osteogenic staining results and (FIG. 3D) quantification of Osteocalcin+ cells. *p<0.05 vs. static control treatment.

FIGS. 4A-4B. Mechanical and pharmaceutical treatments upregulate Sirtuin expression in aged MSCs. MSCs derived from one aged donor were treated with brachial loading and E/E inhibitor and assessed for expression of several proteins in the Sirtuin family. (FIG. 4A) Western blotting results and (FIG. 4B) quantification of Sirtuin protein expression after treatment of MSCs. *p<0.05 vs. static control treatment.

FIGS. 5A-5C. Mechanical conditioning enhances oxidative stress management in aged MSCs. MSCs derived from an 81-year-old female donor were treated with brachial loading and E/E inhibitor for 4 hours per day for 7 days and assessed for proteomic signaling in key pathways related to oxidative stress. (FIG. 5A) Western blotting results and (FIG. 5B) quantification of oxidative stress signaling protein expression after treatment of MSCs. *p<0.05 vs. static control treatment. The MSCs were treated with brachial loading or static control treatments for 4 hours, and flow cytometry for the detection of reactive oxygen species (ROS) was performed. (FIG. 5C) Quantification of median ROS signal intensity. *p<0.05 vs. static control treatment.

FIGS. 6A-6F. Mechanical conditioning enhances DNA damage recognition and repair in aged MSCs. MSCs from an 81-year-old female donor were treated with brachial loading and E/E inhibitor for 4 hours per day for 7 days and assessed for proteomic signaling in key DNA-damage repair pathways. (FIG. 6A) Western blotting results and (FIG. 6B) quantification of DNA damage-related protein expression after treatment of MSCs. *p<0.05 vs. static control treatment. MSCs were treated with brachial loading or static control treatments for 30 minutes, or 4 hours. (FIG. 6C) Immunostaining and (FIG. 6D) quantification of p-γ-H2AX histone foci expression. *p<0.05 vs. 30-minute static control treatment. #p<0.05 vs. 4-hour static control treatment. Brachial conditioning was repeated for 30 minutes, 4 hours, or 4 hours of treatment followed by 20 hours of static treatment. (FIG. 6E) Neutral comet assay images and (FIG. 6F) quantification of DNA damage through calculation of the Olive Tail Moment (OTM) with the OpenComet plugin in FIJI.

FIGS. 7A-7D. Alteration of ATM-mediated DNA damage repair and oxidative stress signaling reduces mechanical protection from senescence and proliferation enhancement. MSCs derived from one aged donor were co-treated with antioxidant N-Acetyl Cysteine (NAC), ATM inhibitor KU55933 (ATMi), and brachial loading. Following the treatments, MSCs were assessed for proliferative function, senescent phenotype population, and proteomic expression in oxidative stress and DNA damage repair proteins. (FIG. 7A) Quantification of BrdU cell proliferation assay after MSCs were co-treated with NAC or KU55933 (ATMi). *p<0.05 vs. static control treatment. (FIG. 7B) Fraction of β-galactosidase-positive cells after MSCs were treated with brachial loading, NAC, or KU55933 (ATMi). *p<0.05 vs. static control treatment. (FIG. 7C) Western blotting results and (FIG. 7D) quantification protein expression after treatment of MSCs. *p<0.05 vs. static control treatment.

FIG. 8. Proposed mechanistic hypothesis of how mechanical conditioning activates oxidative stress and DNA damage signaling to rejuvenate proliferative function and reduce senescence in aged MSCs.

FIG. 9. Proposed biomechanical conditioning regime to improve the efficacy of MSC-based vascular regenerative therapies.

FIGS. 10A-10G. Mechanical treatment of MSCs reverses correlation of gene expression profile with senescence related gene sets. MSCs from one aged donor were conditioned with brachial mechanical loading for 7 days and analyzed with RNA sequencing. (FIG. 10A) Differential gene expression in comparison to the static control group. Genes with statistically significant upregulation (green) or downregulation (red) are shown in color. (FIG. 10B) Clustering analysis of the gene expression in the MSCs for the top significantly differentially expressed genes. Scale: R-log transformed values of raw gene hit counts. (FIG. 10C) Gene ontology analysis of significantly upregulated biological functional processes in the brachial-treated groups in comparison to static-treated control groups. Next, gene set enrichment analyses were performed to compare the gene enrichment in MSCs from one aged donor after control or brachial loading treatment using predefined gene sets for: (FIG. 10D) Genes upregulated in cellular senescence. FDR q-value=0.013. Top 20 genes upregulated in senescence and downregulated after brachial loading. (FIG. 10E) Genes upregulated in elderly donor derived MSCs. FDR q-value=0.003. (FIG. 10F) Genes upregulated in high passage MSCs. FDR q-value=0.001. (FIG. 10G) Genes downregulated in cellular senescence. FDR q-value=0.0001. Top 20 genes downregulated in senescence and upregulated after brachial loading.

FIGS. 11A-11H. Mechanical conditioning of aged MSCs causes altered chromatin accessibility profiles. MSCs from one aged donor were conditioned with brachial mechanical loading for 7 days and analyzed with ATAC sequencing. (FIG. 11A) Enrichment of accessibility chromatin peaks at genomic features. (FIG. 11B) Heatmap of top 20 most differentially upregulated and downregulated ATAC-seq peaks at accessible chromatin regions in brachial vs control treated cells. (FIG. 11C) Reactome pathways associated with highly accessible chromatin regions. (FIGS. 11D-11F) HOMER transcription factor motif enrichment analysis of the upregulated ATAC-seq peaks in brachial treated cells. (FIG. 11D) Transcriptional regulators involving the AP-1 complex. (FIG. 11E) Mechanosensitive signaling factors involving the TGF-β-SMAD2/3 and Yap/Taz-TEAD pathways. (FIG. 11F) Transcriptional regulators of MSC phenotype and resident stem cell proliferation and differentiation potential in skeletal muscle and cardiac tissue. (FIGS. 11G, 11H) Integrated ATAC-seq and RNA-seq analysis. Transcription start sites were mapped to the top 20 differentially enriched peaks for (FIG. 11G) brachial treated cells and (FIG. 11H) control static treated cells. The differential gene expression of these genes for brachial treated samples was compared with differential chromatin accessibility.

FIG. 12. Verification of MSC phenotype in MSCs after biomechanical conditioning during passage 5 and subsequent culture expansion. The negative marker cocktail included antibodies for CD45, CD34, CD11b, CD79A and HLA-DR.

FIGS. 13A-13C. Ponceau staining quantification to confirm equal loading of cell lysate protein.

FIGS. 14A-14B. MSCs from a young donor (24 years old) or aged donor (81 years old) were treated with brachial loading or static control treatment for 4 hours and stained for DAPI. The Nuclear Irregularity Index (NII) plugin in FIJI was used to assess the nuclear morphology of the cells. (FIG. 14A) Sample images processed with the NII plugin. Scale bar=20 μm. (FIG. 14B) Quantification of nuclear morphometric analysis. Nuclei were scored as “Normal”, “Apopotic”, “Senescent”, or “Irregular”. *p<0.05 vs. static control treatment.

FIGS. 15A-15B. MSCs were treated with brachial loading or static control treatments for 4 hours and co-treated with 40 μM Importazole, 2 μM Dooku 1, 3 μM GsMTx4, or DMSO. (FIG. 15A) Immunostaining of p-γ-H2AX histone foci and DAPI. Scale bar=20 μm. (FIG. 15B) Quantification of p-γ-H2AX histone foci expression per nuclear area. *p<0.05 vs. matching static treatment. #p<0.05 vs. static control treatment. †p<0.05 vs. brachial DMSO treatment.

FIG. 16. Images of β-galactosidase staining after MSCs were treated with brachial loading, NAC, or KU55933 (ATMi). Scale bar=100 μm.

FIG. 17. KEGG pathway analysis demonstrating significant upregulation of biosynthesis of unsaturated fatty acids-related pathways in brachial treated MSCs.

FIGS. 18A-18C. Human umbilical vein endothelial cells (HUVECs) at passage 10 were treated with brachial loading for 7 days and assessed for proliferative ability and functional phenotype. (FIG. 18A) Quantification of BrDU cell proliferation assay. (FIG. 18B) Quantification of fraction of β-galactosidase-positive cells. *p<0.05 vs. static control treatment. (FIG. 18C) Quantification of the cumulative doubling of the treated cells during long-term culture expansion.

### DETAILED DESCRIPTION

Mesenchymal stem cells are a promising cell type for autologous regenerative therapies, but clinical trials have largely yielded inconsistent and disappointing results. A major reason for these limitations is cellular senescence, which is a state in which a cell loses its capacity to replicate and/or loses its therapeutic and functional properties due to cellular aging. Cellular senescence is caused by poor donor health (Loomans et al., 2004; Vecellio et al., 2014), advanced age (Kuang et al., 2015; Lin et al., 2019), and the extensive in vitro expansion required to generate an adequate number of cells for therapeutic applications (Zhou et al., 2020). Senescence also causes MSCs to lose their multipotency, genetic stability, and immunomodulatory functions, drastically limiting their efficacy in autologous therapies. Senescent MSCs are known to secrete pro-inflammatory factors that worsen tissue regeneration and remodeling and spread the senescence to other cells in their microenvironment.

Mechanically conditioning MSCs derived from aged donors enhances their short and long-term proliferative function, improves their multipotency, and protects the cells from advancing senescence, resulting in a lower population of senescent cell phenotypes following the treatment. Brachial mechanical loading in co-treatment with an EGFR/ErbB-2/4 (E/E) synergistically enhances the angiogenic properties of MSCs (Lee et al., 2021a). However, brachial loading alone is superior in enhancing aged MSC proliferative function, expansion capacity, and multipotent properties. In this approach, brachial loading alone is first used to improve the efficiency of MSC culture expansion. Once a sufficient population of healthy cells are generated, the E/E inhibitor is added as a cotreatment to drive MSC differentiation into vascular phenotypes with improved regenerative properties before delivery to patients with peripheral artery disease and ischemia. The brachial loading as a first step to MSC expansion could also be useful in many other applications, if later followed by differentiation into other phenotypes. By conditioning the cells for a short time each day for one week, they have enhanced expansion capacity including increased proliferation and expansion in the long term. For instance, growing 10,000 MSCs out for 7 passages using prior methods could expand these into around 1 million cells. Using the methods described herein, 10,000 MSCs can be expanded into 9.8 million cells.

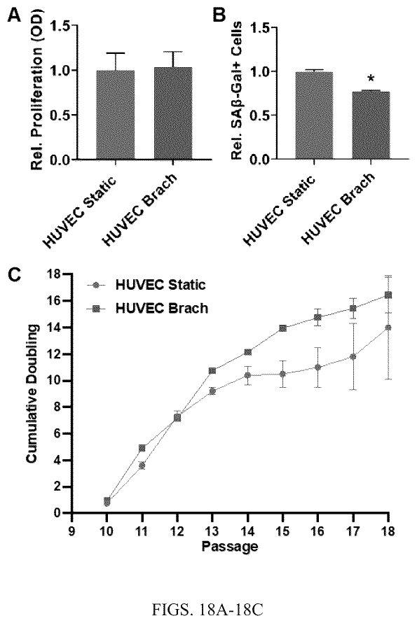

Mechanical loading may be universally applied to enhance the expansion of various cell cultures. According to a computational model (Miles et al., 2023), mechanical strains within an optimal range may exert forces on chromatin, potentially leading to the opening of DNA without DNA damage. Notably, from the model of cell stretching, brachial loading appears to have a broader range where it can facilitate DNA opening without causing damage. This empirical evidence combined of these studies with these models suggests that a range of cell types might similarly benefit from such biophysical interventions to augment their culture expansion, diminish senescence, and stimulate DNA damage repair mechanisms. To explore this hypothesis further, high-passage human umbilical vein endothelial cells (HUVECs) at passage 10 were treated with brachial loading. Their proliferation, β-galactosidase expression, and capacity for long-term culture expansion were measured. While brachial loading did not enhance short-term DNA synthesis, it significantly decreased the number of β-galactosidase positive cells and markedly improved the cumulative doubling rate during extended in vitro culture (FIG. 18).

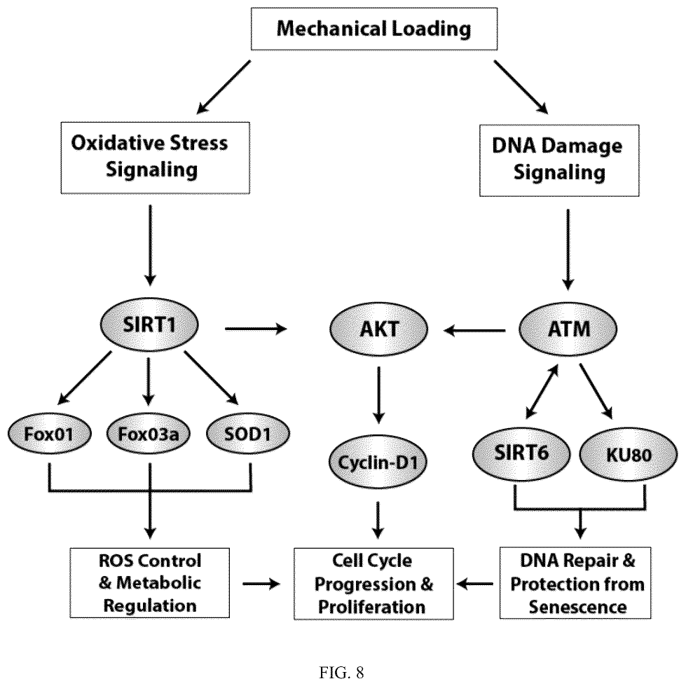

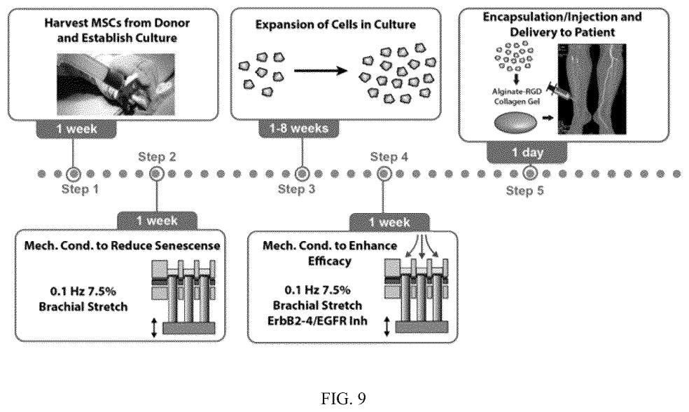

Brachial mechanical loading results in the upregulation of several Sirtuin proteins, which are known to be crucial regulators of aging. Several current clinical trials are investigating the anti-aging potential of pharmacological SIRT1 activators such as Resveratrol (Pyo et al., 2020) and Metformin (Chalasani et al., 2018), although results remain inconclusive in human subjects. Sirtuin proteins may alter key pathways of senescence, oxidative stress management and DNA damage repair, to rejuvenate the functionality of aged MSCs. Previous work has shown that SIRT6 functions as a DNA damage sensor and activates DNA damage signaling through the recruitment and phosphorylation of ATM and γ-H2AX histone foci (Onn et al., 2020). The activated ATM in turn phosphorylates Akt to promote cell survival and proliferation (Halaby et al., 2008), which is in agreement with the results of the mechanistic studies provided herein (FIG. 8).

Mechanical conditioning of aged MSCs results in the enhanced recognition and repair of DNA double-strand-breaks. Enhanced DNA damage repair was not dependent on Piezo1-mediated mechanosensitive signaling and only partially dependent on import of nuclear proteins through importin-1. In addition, mechanical loading caused enhanced recognition of DNA damage leading its repair over time. Together, these findings suggest a biophysical mechanism in which mechanical loading reveals occult DNA damage that can then be repaired. Mechanical conditioning also resulted in the broad activation of antioxidant proteins such as SOD1, FOX01, FOX03a and FOX04, and DNA damage repair proteins such as KU80 and ATM. Mechanical conditioning also reduced the buildup of reactive oxygen species and resulted in rapid repair of DNA damage. When oxidative signaling was altered through the co-treatment of aged MSCs with the antioxidant N-Acetyl Cysteine (NAC) and brachial loading, a reduction of proliferative enhancement was observed. Conversely, when DNA damage repair signaling was altered through the co-treatment of ATM inhibitor KU55933 with brachial loading, a reduction of senescence-protective effects was observed, resulting in a larger phenotype of β-Galactosidase+ senescent cell phenotypes. Together, these results suggest that mechanical conditioning relies upon altered signaling in both key pathways of senescence to rejuvenate specific aspects of the cells' functionality.

Several studies have targeted the elevated reactive oxygen species (ROS) and impaired antioxidant mechanisms that are characteristic of senescent MSCs, using gene vectors to overexpress the Sirtuin proteins (Meng et al., 2020; Zhang et al., 2018), antioxidant pharmaceutical treatment (Lee et al., 2016; Park et al., 2017), or conditioning with extracellular exosomes (Khanh et al., 2020). However, additional studies have shown that high doses of antioxidant expression can lead to DNA damage and induce premature senescence, and there remains a need to further evaluate anti-aging antioxidant potential (Kornienko et al., 2019). Other studies have shown that over-activation of the DNA damage repair network can transiently halt cell cycle progression in aged MSCs and persistence of DNA damage results in activation of tumor suppressor functions to induce senescence (Zhang et al., 2009; Ciccia & Elledge, 2010). Mechanical conditioning led to upregulated antioxidant expression and enhanced DNA damage repair, suggesting that there is a crucial homeostatic balance of signaling in and between these pathways in the rejuvenation of senescence. Therefore, while highly targeted attempts to improve the antioxidant or DNA damage repair response genetically or pharmacologically may cause unintended consequences, mechanical treatment resulted in the broad enhancement of these pathways in aged MSCs to restore their functionality. Taken together, these findings provide a practical method to enhance the regenerative function of MSCs in aged patients.

While this technique has been tested in the rejuvenation of aged MSCs, it is expected that other cell types would receive similar functional enhancements. Senescence is not unique to MSCs, but also limits cell therapies for other diseases including cancer (e.g., CAR-T, T cells), chronic kidney disease (e.g., epithelial cells, endothelial cells), vascular disease and tissue engineered grafts (e.g., smooth muscle cells, endothelial cells), metabolic disorders, and inflammatory conditions (Huang et al., 2022). While the specific use of the treated cells would vary for each of these therapeutic indications, all are currently limited by the diminished proliferation, expansion capacity, and regenerative potential of the senescent cells.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

The term “cell population” is used herein to refer to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and depleted of one or more cell types.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs.”

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell is an embryonic stem cell derived from the inner cell mass of a blastocyst. In other embodiments, the pluripotent stem cell is an induced pluripotent stem cell derived by reprogramming somatic cells.

The term “differentiation” refers to the process by which an unspecialized cell becomes a more specialized type with changes in structural and/or functional properties. The mature cell typically has altered cellular structure and tissue-specific proteins.

As used herein, “undifferentiated” refers to cells that display characteristic markers and morphological characteristics of undifferentiated cells that clearly distinguish them from terminally differentiated cells of embryo or adult origin.

An “isolated” cell has been substantially separated or purified from others cells in an organism or culture. Isolated cells can be, for example, at least 99%, at least 98% pure, at least 95% pure or at least 90% pure.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

A “therapeutically effective amount” used herein refers to the amount of a compound that, when administered to a subject for treatment of a disease or condition, is sufficient to effect such treatment.

A “brachial waveform” as used herein refers to a waveform with 2 or more peaks during one cycle. A brachial waveform may mimic the distension of arteries during the cardiac cycle. For example, a brachial waveform may consist of a strain waveform that has two peaks in one cycle, with the first peak being higher than the second peak.

### II. Peripheral Vascular Disease

Peripheral vascular disease (PVD) can result from atherosclerotic occlusion of the blood vessels, particularly in limbs and areas distal from the heart, resulting in diminished blood flow and insufficient oxygen perfusion to tissues in the vicinity of and downstream from the occlusion. PVD is frequently manifested in the iliac blood vessels, femoral and popliteal blood vessels, and subclavian blood vessels, and its effects can be exacerbated by thrombi, emboli, or trauma. It is estimated that approximately 8 to 12 million individuals in the United States, especially among the elderly population and those with diabetes, are afflicted with PVD.

Common symptoms of PVD include cramping in the upper and lower limbs and extremities, numbness, weakness, muscle fatigue, pain in the limbs and extremities, hypothermia in the limbs and extremities, discoloration of the extremities, dry or scaly skin, and hypertension. The most common symptom is claudication or feelings of pain, tightness, and fatigue in muscles downstream of the occluded blood vessel that occurs during some form of exercise such as walking, but self-resolve after a period of rest.

In terms of pathophysiology, the occluded blood vessels cause ischemia of tissues at the site of and distal to the obstruction. This ischemia is generally referred to as peripheral ischemia, meaning that it occurs in locations distal to the heart. The severity of the ischemia is a function of the size and number of obstructions, whether the obstruction is near a muscle or organ, and whether there is sufficient redundant vasculature. In more severe cases, the ischemia results in death of the affected tissues, and can result in amputation of affected limbs, or even death of the patient.

Current methods for treatment of more severe cases of PVD include chemotherapeutic regimens, angioplasty, insertion of stents, reconstructive surgery, bypass grafts, resection of affected tissues, or amputation. Unfortunately, for many patients, such interventions show only limited success, and many patients experience a worsening of the conditions or symptoms.

An emerging strategy for treating ischemia is to use growth factors, gene therapies, or regenerative cell types to stimulate tissue repair via the generation of new blood vessels. (Veith, A. P., et al. (2019). “Therapeutic strategies for enhancing angiogenesis in wound healing.” Advanced Drug Delivery Reviews146: 97-125.) (Carmeliet, P. and M. Baes (2008). “Metabolism and Therapeutic Angiogenesis.” New England Journal of Medicine358 (23): 2511-2512.)

Presently, there is interest in using either stem cells, which can divide and differentiate, or muscles cells from other sources, including smooth and skeletal muscles cells, to assist the in the repair or reversal of tissue damage. Transplantation of stem cells can be utilized as a clinical tool for reconstituting a target tissue, thereby restoring physiologic and anatomic functionality. The application of stem cell technology is wide-ranging, including tissue engineering, gene therapy delivery, and cell therapeutics, i.e., delivery of biotherapeutic agents to a target location via exogenously supplied living cells or cellular components that produce or contain those agents. The identification of stem cells has stimulated research aimed at the selective generation of specific cell types for regenerative medicine.

One obstacle to realization of the therapeutic potential of stem cell technology has been the difficulty of obtaining sufficient numbers of stem cells. A reliable, well-characterized and plentiful supply of substantially homogeneous populations of such cells having the ability to differentiate into an array of skeletal muscle, pericyte, or vascular lineages would be an advantage in a variety of diagnostic and therapeutic applications for skeletal muscle repair, regeneration, and improvement, for the stimulation and/or support of angiogenesis, and for the improvement of blood flow subsequent to a peripheral ischemic event, particularly in PVD patients.

The pharmaceutical compositions provided herein may be administered at sites of peripheral ischemia. The pharmaceutical composition may be administered locally. The pharmaceutical composition may be administered by injection, infusion, a device implanted in a patient, or by implantation of a matrix or scaffold containing the pharmaceutical composition. The pharmaceutical composition may be administered by injection into interstitial spaces so as not to enter directly into circulation. The isolated population of cells may be induced in vitro to differentiate into a skeletal muscle, vascular muscle, pericyte or vascular endothelium lineage prior to administration. The population of cells may also be genetically engineered to produce a gene product that promotes treatment of peripheral vascular disease. Optionally, the composition further comprises an agent selected from the group consisting of an antithrombogenic agent, an immunosuppressive agent, an immunomodulatory agent, a pro-angiogenic, an antiapoptotic agent and mixtures thereof.

Intra-arterial administration (IA) involves delivery of the pharmaceutical compositions into at least one artery. In an IA infusion, the infusion is typically divided into several arteries in the legs, e.g., the left and right common femoral arteries, but is sometimes administered into a single artery. The infusion can be administered for about 1 minute, 1 to 5 minutes, 10 to 20 minutes, or 20 to 30 minutes into each artery in both legs. The infusion can be repeated from time to time to achieve or sustain the predicted benefit. The timing for repeat administration is based on the patient's response as measured by symptoms and hemodynamic measures.

### III. Therapeutic Compositions

Providing a therapy or “treating” refers to indicia of success in the treatment or amelioration of an injury, disease or condition, including any objective or subjective parameter such as abatement, remission, diminishing of symptoms of making the injury, disease or condition more tolerable to the patient, slowing the rate of degeneration or decline, making the final point of degeneration less debilitating, or improving a patient's physical or mental well-being. Those in need of treatment include those already with the disease or condition as well as those prone to have the disease or condition or those in whom the disease or condition is to be prevented. Preferred subjects for treatment include animals, most preferably mammalian species, such as humans and domestic animals such as dogs, cats, and the like, subject to the disease and other conditions. A “patient” refers to a subject, preferably mammalian (including human). Where the specification indicates that a number of cells are to be administered, a person of ordinary skill in the art will understand that these are approximate values.

In some embodiments, the dosage of the composition encompassing a therapeutically effective amount of mesenchymal stem cells ranges from 1.0×10³-1.0×10⁸ cells/kg (weight). In some embodiments, the dosage ranges from 1.0×10⁴-1.0×10⁷ cells/kg (weight). In some embodiments, about 1.0×10³ cells/kg, about 1.0×10⁴ cells/kg, about 1.0×10⁵ cells/kg, about 1.0×10⁶ cells/kg, about 1.0×10⁷ cells/kg or about 1.0×10⁸ cells/kg are administered.

The dosage of the composition may vary depending on patient's weight, age, sex and symptoms, the dosage form of the composition to be administered, a method of administering the composition, and so on. The frequency of administration may range from one to several times. There may be one or more administration sites. The dosage per kg for non-human animals may be the same as that for human, or can be converted from the above-described dosage, for example, based on the volume ratio (for example, average value) between the diseased tissue of the human and animal subjects. Animals to be treated according to the present invention include human and other desired mammals, specific examples of which include humans, monkeys, mice, rats, rabbits, sheep, horses, cats, cows and dogs.

In accordance with the present invention, the diseases or conditions can be treated or prevented by intravenous administration of the mesenchymal stem cells described herein. In some embodiments, about 20 million, about 40 million, about 60 million, about 80 million, about 100 million, about 120 million, about 140 million, about 160 million, about 180 million, about 200 million, about 220 million, about 240 million, about 260 million, about 280 million, about 300 million, about 320 million, about 340 million, about 360 million, about 380 million, about 400 million, about 420 million, about 440 million, about 460 million, about 480 million, about 500 million, about 520 million, about 540 million, about 560 million, about 580 million, about 600 million, about 620 million, about 640 million, about 660 million, about 680 million, about 700 million, about 720 million, about 740 million, about 760 million, about 780 million, about 800 million, about 820 million, about 840 million, about 860 million, about 880 million, about 900 million, about 920 million, about 940 million, about 960 million, or about 980 million cells are injected intravenously. In some embodiments, about 1 billion, about 2 billion, about 3 billion, about 4 billion or about 5 billion cells or more are injected intravenously. In some embodiments, the number of cells ranges from between about 20 million to about 4 billion cells, between about 40 million to about 1 billion cells, between about 60 million to about 750 million cells, between about 80 million to about 400 million cells, between about 100 million to about 350 million cells, and between about 175 million to about 250 million cells.

In some embodiments, a single intravenous administration is sufficient, while in other embodiments, multiple intravenous administrations are performed, such as 2, 3, 4, 5, 6, 7, 8, 9 10, 11, 12, 13, 14, 15, 16, 17, 18, 19, or 20 intravenous administrations of the mesenchymal stem cells. The treatment interval(s) can be spaced such that an administration follows a prior administration by one day, 2 days, 3 days, 4 days, 5 days, 6 days, one week, 1½-2 weeks, 3 weeks, one month, or 2-3 months, 6 months, one year, or two years or longer. In some embodiments, the treatment interval is spaced in accordance with the progression of the patient's improvement or response to treatment. For example, in some embodiments, a first treatment is administered followed by a second treatment one week later, followed by a third treatment one month later, followed by a fourth treatment 6 months later.

The therapeutic methods of the present invention can be conducted alone or in combination with other standard or advanced methods or pharmaceutical treatments.

The therapeutic composition of mesenchymal stem cells for use in the methods of the present invention can comprise pharmaceutically acceptable carriers and/or additives. Examples thereof include sterilized water, physiological saline, a standard buffer (e.g., phosphoric acid, citric acid, or other organic acids), a stabilizer, salt, an antioxidant (e.g., ascorbic acid), a surfactant, a suspending agent, an isotonic agent, or a preservative. As used herein, the term “base” refers to a base solution in which the mesenchymal stem cells in the cell therapeutic composition are suspended. In some embodiments, physiological saline, phosphate buffered saline or Hartrnan-D (Choongwae Pharma Corp.) is used as the base solution.

In some embodiments, the cell therapeutic composition is prepared in a dosage form suitable for injection. In some embodiments, the mesenchymal stem cells are dissolved (suspended) in a pharmaceutically acceptable aqueous solution, or frozen in a solution state. The kit of the present invention may further comprise a desired pharmaceutically acceptable carrier that can be used to suspend or dilute the mesenchymal stem cells. Examples of such a carrier include distilled water, physiological saline, PBS and the like.

The composition for use in the present invention can contain a pharmaceutically acceptable carrier or excipient, or any necessary stabilizer or adsorption-preventing agent to provide a pharmaceutical preparation that is suitable for administration to humans or animals. The composition of the present invention can be formulated in the form of an injectable solution (e.g., injection solutions for subcutaneous, intradermal, intramuscular, intravenous and intraperitoneal injection). In some embodiments, upon the injection of the composition of mesenchymal stem cells, an analgesic agent, which can relieve pains, may be used.

The cell therapeutic composition of mesenchymal stem cells for use in the present invention can be filled into a syringe, a device, a cryovial in which cells can be frozen, or a pyrogen-free glass vial comprising rubber stoppers and aluminum caps, which contains liquid drugs.

The cell therapeutic composition of mesenchymal stem cells for use in the present invention can, if necessary, contain at least one selected from among suspending agent, solubilizing agents, stabilizers, isotonic agents, preservatives, adsorption-preventing agents, surfactants, diluents, vehicles, pH-adjusting agents, analgesic agents, buffering agents, sulfur-containing reducing agents and antioxidants, depending on the administration mode or formulation thereof.

Examples of the suspending agents may include methylcellulose, Polysorbate 80, hydroxyethylcellulose, gum acacia, gum tragacanth powder, sodium carboxymethylcellulose, polyoxyethylene sorbitan monolaurate, etc. The solubilizing agents include polyoxyethylene hydrogenated castor oil, polysorbate 80, nicotinamide, polyoxyethylene sorbitan monolaurate, Macrogol and castor oil fatty acid ethyl esters. The stabilizers include dextran 40, methylcellulose, gelatin, sodium sulfite, sodium metasulfite, etc. Examples of the isotonic agents are D-mannitol and sorbitol.

Examples of the preservatives include methyl parahydroxybenzoate, ethyl parahydroxybenzoate, sorbic acid, phenol, cresol, and chlorocresol. Examples of the adsorption preventing agents include human serum albumin, lecithin, dextran, ethylene oxide-propylene oxide copolymer, hydroxypropylcellulose, methylcellulose, polyoxyethylene hydrogenated castor oil, and polyethylene glycol.

The sulfur-containing reducing agents include N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, and sulfhydryl-containing compounds such as thioalkanoic acid having 1 to 7 carbon atoms.

The antioxidants include, for example, erythorbic acid, dibutylhydroxytoluene, butylhydroxyanisole, [alpha]-tocopherol, tocopherol acetate, L-ascorbic acid and salts thereof, L-ascorbyl palmitate, L-ascorbyl stearate, sodium bisulfite, sodium sulfite, triamyl gallate, propyl gallate or chelating agents such as disodium ethylenediamine tetraacetate (EDTA), sodium pyrophosphate and sodium metaphosphate. The cryopreservatives include, for example, DMSO, glycerol, etc.

Furthermore, in some embodiments, the cell therapeutic composition of mesenchymal stem cells for use in the methods of the present invention can comprise conventional additives, such as inorganic salts, including sodium chloride, potassium chloride, calcium chloride, sodium phosphate, potassium phosphate and sodium hydrogen carbonate, and organic salts, including sodium citrate, potassium citrate and sodium acetate.

In some embodiments, sucrose or albumin is added to the mesenchymal stem cells to improve stability, prior to cold storage of the cells. In some embodiments, the cells are combined with physiological saline, sucrose, albumin and cryopreservative DMSO prior to freezing and cold storing the cells.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Mechanical Conditioning Reduces Senescence Related Gene Expression in MSCs from Young Donors

The inventors have developed a system that enables the application of mechanical stretch in a high throughput format. Using this system, the inventors have demonstrated methods for using specific mechanical loading regimes and pharmacological compounds to enhance the regenerative capacity of MSCs from young donors for treating peripheral ischemia (Lee et al., 2021a). In these studies, maximum angiogenic signal responsiveness was achieved with a mechanical loading regime of 4 hours per day for 7 days of 7.5% maximal strain at 0.1 Hz frequency of loading using a physiological waveform similar to stretch of the brachial artery during the cardiac cycle (brachial loading) (Lee et al., 2013). In addition, 1 μM EGFR/ErbB-2/4 (E/E) inhibitor as a cotreatment with loading led to a dramatic increase in MSCs that expressed the markers for both endothelial cells and pericytes, and the resulting cells had enhanced angiogenic activity in a tube formation assay (Lee et al., 2021a).

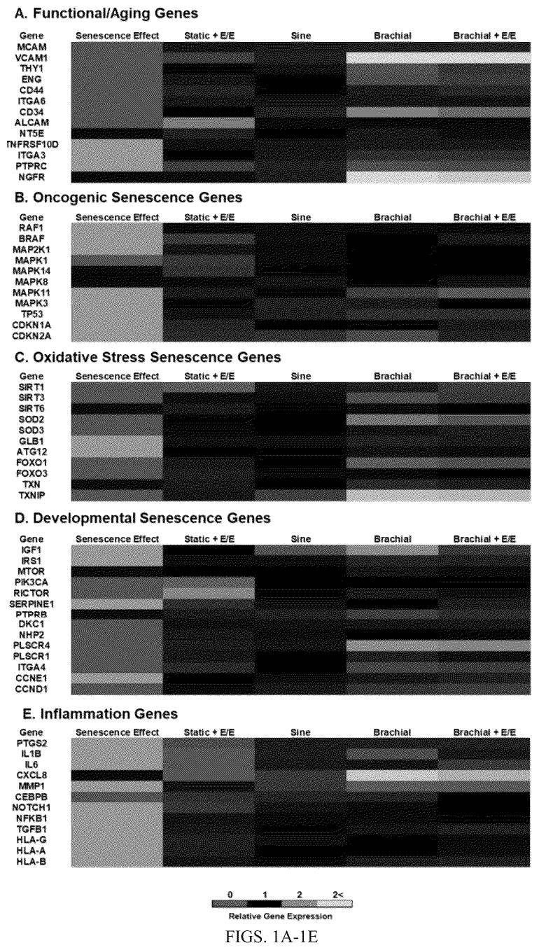

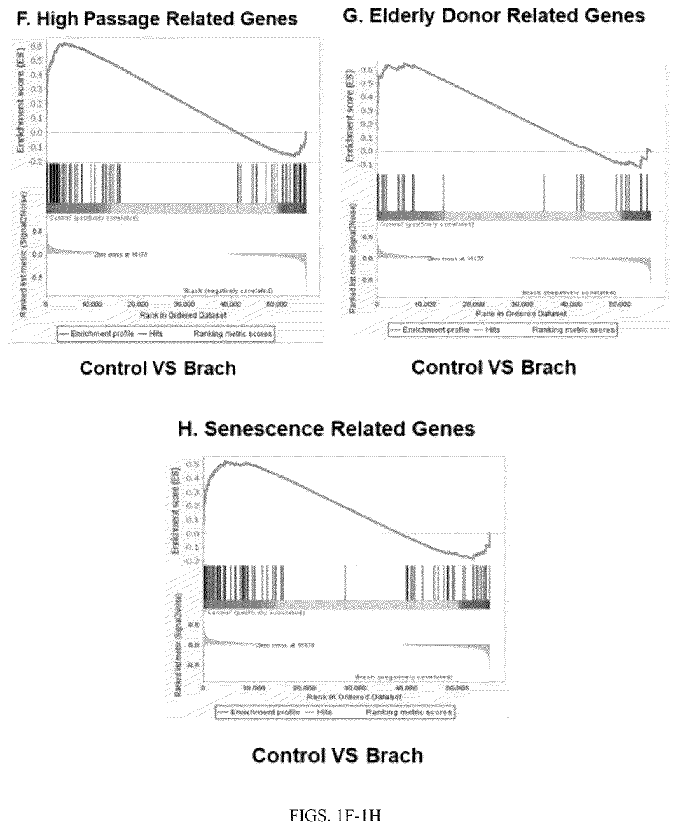

To investigate the treatments' effects on gene expression patterns that are correlated with senescence in MSCs, MSCs were obtained from three young, healthy donors, and conditioned with brachial loading and E/E inhibitor. Following the treatments, RNA-seq analysis was performed on the cells, and the expression of several key gene sets related to various mechanisms of senescence in MSCs was evaluated. These gene sets were identified through a thorough investigation of the literature as key mediators of MSC senescence, and the expression pattern of each gene as it pertains to MSC senescence was documented (Zhou et al., 2020; Brandl et al., 2011; Turinetto et al., 2016; Ozkul & Galderisi, 2016; Fridman & Tainsky, 2008; Ki et al., 2014; Cakouros & Gronthos, 2019; Peffers et al., 2016). The analysis found that the treatments reversed the effects implicated by senescence on genes related to age-related senescence, oncogenic-related senescence, stress-related senescence, developmental-related senescence, and inflammation-mediated senescence (FIG. 1). Next, a gene set enrichment analysis (GSEA) was performed on the RNA-seq data. Enrichment scores comparing the genes expressed between the mechanically conditioned cells and control cells were calculated for gene sets upregulated in high passage MSCs (GSE137186) (Salerno et al., 2020), genes upregulated in elderly donor-derived MSCs (E-MTAB-4879) (Peffers et al., 2016), and genes upregulated in senescent cells (M27188) (Fridman & Tainsky, 2008). In all three senescence-related gene sets, a reduction in senescence related genes with brachial mechanical loading was observed (FIG. 1). These findings suggest that brachial loading may cause a broad reversal of gene expression patterns that are correlated with senescence in MSCs, supporting the hypothesis that mechanical conditioning rejuvenates MSCs and protects them from senescence.

### Example 2—Mechanical Conditioning Enhances Proliferation in MSCs from Aged Donors

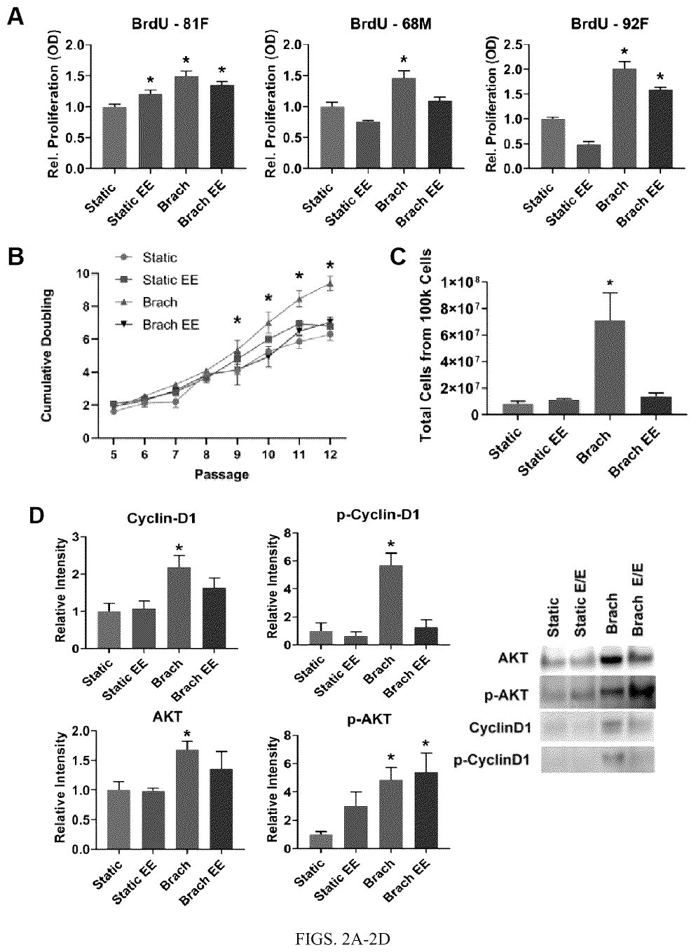

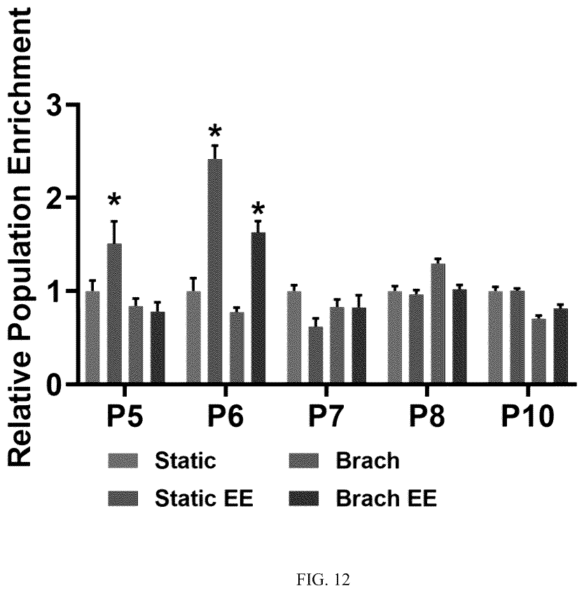

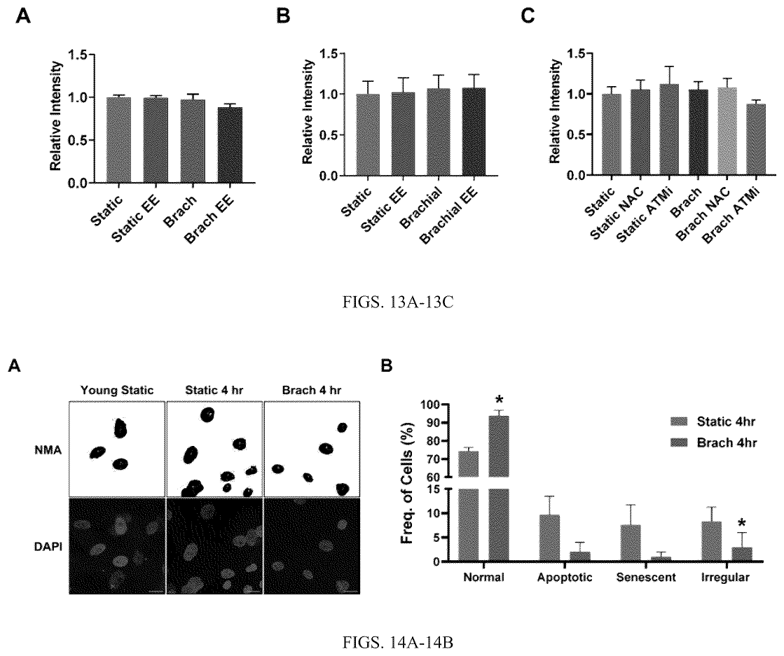

To test if mechanical loading could improve the proliferative function of aged MSCs, MSCs from three donors (ranging from 68-92 years of age) were conditioned with a brachial waveform at a maximal strain of 7.5% for 7 days for 4 hours per day in combination with 1 μM EGFR/ErbB-2/4 (E/E) inhibitor. After the treatments, cells were evaluated for DNA synthesis with a bromodeoxyuridine (BrdU) proliferation assay. Brachial loading increased DNA synthesis in cells from all three donors, and brachial+E/E inhibitor treatment increased DNA synthesis in two of three donor lines, relative to the static control (FIG. 2A). Additionally, treated cells from an 81-year-old female donor were collected and seeded into standard culture plates and passaged until they reached full senescence and failed to continue proliferating. Cells were counted at each passage, and brachial loaded cells were shown to maintain a significantly faster proliferative rate during passages 9-12, while static groups reached comparative growth arrest (FIG. 2B). To illustrate the functional meaning of these findings, the number of cells that would be produced from 100,000 cells was calculated under the different treatments during the expansion (FIG. 2B). Treatment with the brachial loading would lead to roughly 70 million cells while the other treatments would lead to generation of approximately 8 million cells. To verify that biomechanically treated MSCs retained their phenotype after the treatment and during subsequent culture expansion, testing for the MSC phenotype of the cells was performed using flow cytometry (FIG. 12). Treated cells were evaluated for expression of proliferation-related proteins by Western blotting (FIG. 2D). A Ponceau stain was run to confirm equal loading of cell lysate protein in addition to the non-phosphorylated protein blots (FIG. 13A). Results showed significant upregulation of AKT (pan), p-AKT (Ser473), cyclin D1, and p-cyclin D1 (Thr286) with mechanical treatment alone.

### Example 3—Mechanical Conditioning Enhances Multipotency of Aged MSCs During Cell Culture Expansion

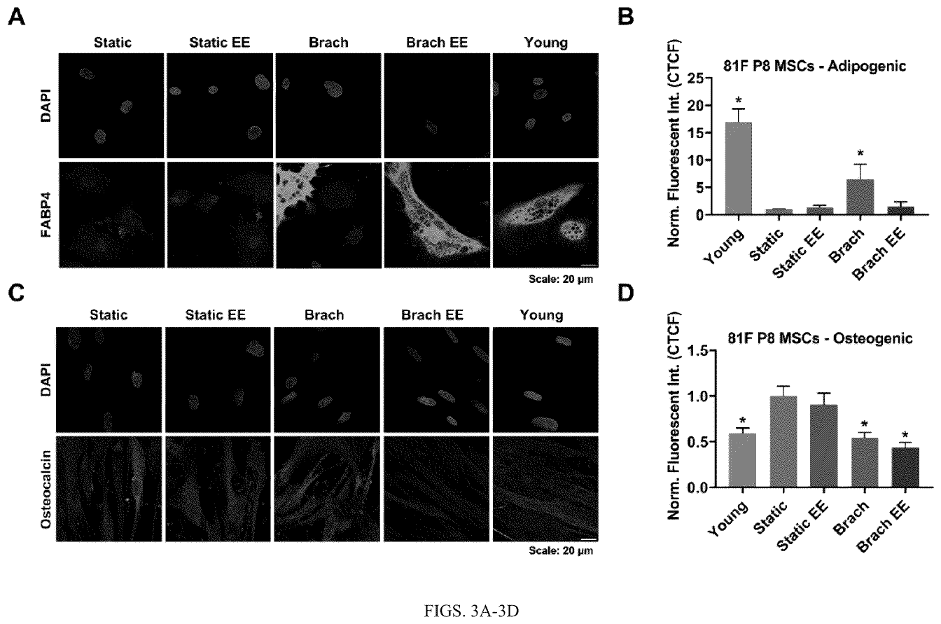

Senescent MSCs have a reduced capacity to differentiate into other cell phenotypes, which limits their clinical utility (Siegel et al., 2013). To test if mechanical conditioning improves the multipotency of aged MSCs, brachial loading and E/E inhibitor treatments were repeated on P5 MSCs from an 81-year-old female donor. Following the treatments, the MSCs were expanded in culture until P8, then evaluated for adipogenic and osteogenic differentiative potential with the hMSC functional identification kit (R&D Systems, SC006). A young MSC line from a 24-year-old female donor was also expanded until P8 and included as a control. The rationale for using cells at P8 was that fully senescent MSCs are expected to completely lose the ability to undergo functional tri-linage differentiation, and studies have shown that an almost complete loss in multipotency occurs around P9/P10 for cells derived from old donors (60+ years) (Zaim et al., 2012). Thus, cells were tested just prior to the theoretic point of complete senescence to determine if the mechanical and pharmaceutical conditioning treatments can improve impaired multipotency. Following differentiation, cells were fixed and stained with FABP4 to evaluate adipogenic differentiation and Osteocalcin to evaluate osteogenic differentiation (FIG. 3). Results showed a significant increase in adipogenic differentiated cells with brachial loading, in comparison to the static control (FIGS. 3A, 3B). A decrease in osteogenic differentiation was observed with brachial loading, but this matched the trend of the young control line, suggesting that the mechanical and pharmaceutical conditioning treatments may cause the cells to adopt a phenotype more similar to cells from younger donors (FIGS. 3C, 3D). Furthermore, other studies have shown that the expression of osteogenic genes including osteocalcin tend to increase during MSC aging and ex vivo expansion, suggesting that the mechanical and pharmaceutical conditioning treatment may be inhibiting or reversing this process (Cakouros & Gronthos, 2019).

### Example 4—Mechanical Conditioning Upregulates Sirtuin Expression in Aged MSCs

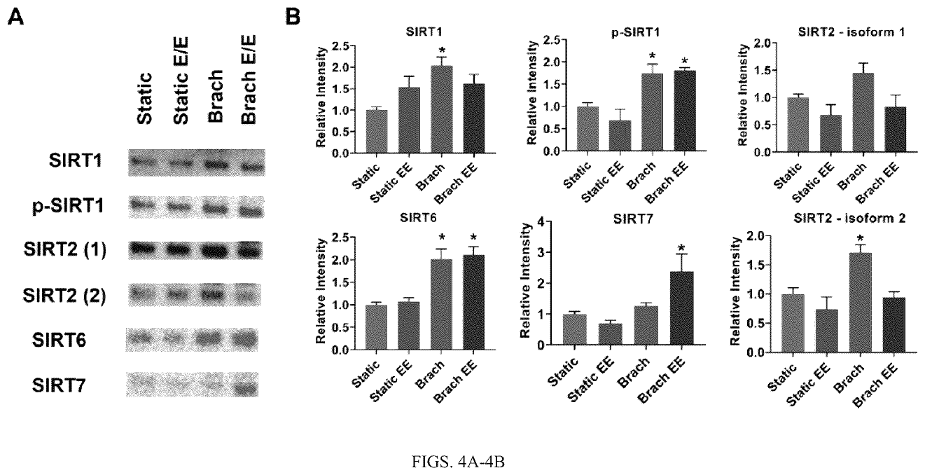

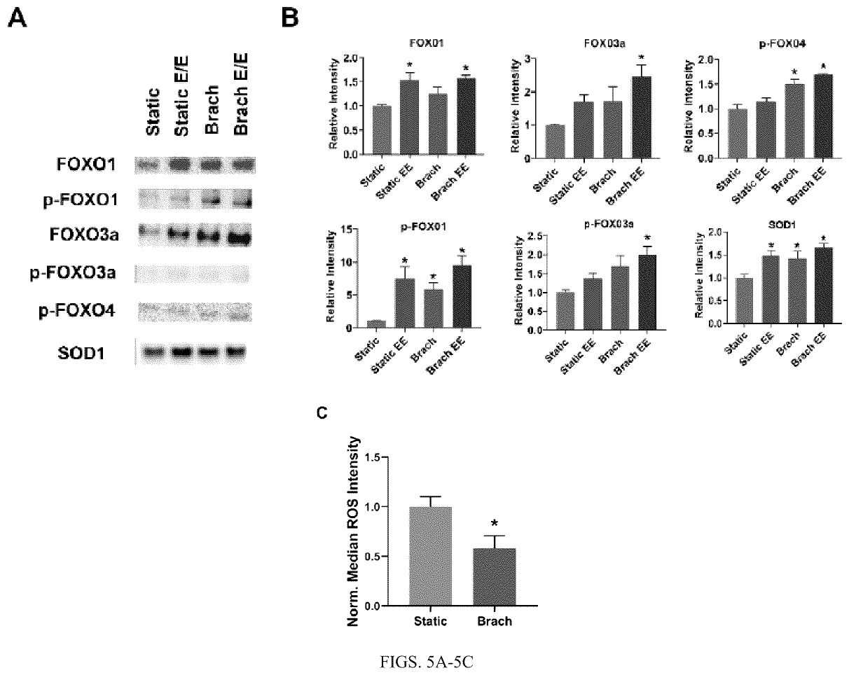

The Sirtuin family of proteins are nicotinamide dinucleotide (NAD+) dependent enzyme deacylases that regulate many cellular processes, including DNA repair, differentiation, metabolism, inflammation, and aging (Lee et al., 2019). Several members of the Sirtuin family have also been implicated as major regulators of multiple senescence pathways (Cakouros & Gronthos, 2019; Onn et al., 2020). To investigate the effects of mechanical and pharmaceutical conditioning on Sirtuin expression, MSCs from an 81-year-old donor were treated with brachial loading and E/E inhibition and Western blotting performed for several proteins in the Sirtuin family. Results showed significant upregulation of SIRT1, p-SIRT1, a SIRT2 isoform, and SIRT6 with brachial loading, and upregulation of p-SIRT1, SIRT6, and SIRT7 with brachial+E/E treatment, in comparison to the static control (FIGS. 4A, 4B). Ponceau stain was run to confirm equal loading of cell lysate protein (FIG. 13A). This broad activation of Sirtuin expression suggests that the mechanical and pharmaceutical treatments are altering signaling in age or senescence-related pathways, possibly to rejuvenate the proliferative and multipotent functions of the MSCs.

### Example 5—Mechanical Conditioning Enhances Oxidative Stress Management in Aged MSCs

The effective management of oxidative stress and repair of DNA damage are critically important mechanisms for preventing senescence in MSCs (Banimohamad-Shotorbani et al., 2020; Brandl et al., 2011; Yu et al., 2018). To test if the mechanical and pharmaceutical treatments could alter these mechanisms, the mechanical and pharmaceutical treatments were repeated on MSCs from an aged donor (age 81), which were evaluated for protein expression with Western blotting in pathways related to oxidative stress (FIGS. 5A, 5B). A Ponceau stain was included to confirm equal loading of cell lysate protein (FIG. 13B). Results showed significant upregulation of FOX0 transcription factors, which are critical mediators of the cellular response to ROS in MSCs (Liang & Ghaffari, 2013; Atashi et al., 2015; Kim et al., 2012). Brachial loading or brachial+E/E inhibitor treatment caused an upregulation of FOX01, p-FOX01, FOX03a, p-FOX03a, and p-FOX04. Additionally, the treatments caused significantly upregulated expression of the antioxidant enzyme SOD1 (Gharibi et al., 2014). (FIGS. 5A, 5B). Next, the MSCs were conditioned with brachial loading or static control treatment for 4 hours and stained for oxidative stress with the CellROX Deep Red Reagent (ThermoFisher). The Sytox Blue dye (ThermoFisher) was included to distinguish dead cells from viable cells and flow cytometry was performed. Mechanically conditioned cells had a significantly lower expression of reactive oxygen species (ROS) in comparison to the static control (FIG. 5C). These results indicate that the brachial loading treatment alters the cellular response to oxidative stress signaling and mitigates ROS buildup. Oxidative stress often precedes senescence, leading to lessened proliferation, accelerated growth arrest, and DNA damage-related genetic instability (Brandl et al., 2011; Benameur et al., 2015).

### Example 6—Mechanical Conditioning Enhances the Recognition and Repair of DNA Damage in Aged MSCs

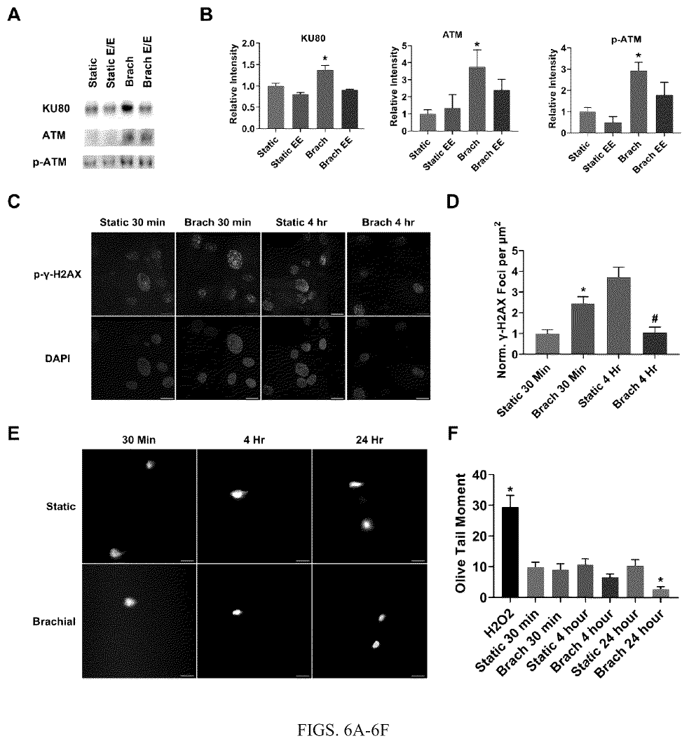

Long term culture of MSCs impairs the ATM-dependent recognition of DNA breaks, leading to the buildup of DNA damage and genetic instability (Hladik et al., 2019). To investigate the effects of mechanical loading on DNA damage repair signaling, the mechanical and pharmaceutical treatments were repeated on MSCs from an aged donor (age 81), which were evaluated for protein expression by Western blotting in pathways related to DNA damage repair (FIG. 6A, 6B). A Ponceau stain was run to confirm equal loading of cell lysate protein (FIG. 13B). Results showed significant upregulation of DNA-damage repair proteins KU80 (Oliver et al., 2013), ATM (Lee & Paull, 2021), and p-ATM (Tang et al., 2019) with mechanical treatment (FIGS. 6A, 6B).

To investigate the downstream effects of mechanically altered DNA damage repair signaling, MSCs from the 81-year-old donor were treated with brachial loading or static conditions for 30 minutes, and 4 hours. Next, immunostaining for p-γ-H2AX DNA double-strand break repair foci was performed. The histone H2AX makes a critical contribution to genetic stability through signaling the recognition of DNA damage events and acting as a foundation for the assembly of repair machinery (Pinto & Flaus, 2010). Brachial treatment for 30 minutes resulted in a significantly higher count of p-γ-H2AX foci compared to the static control at the same time point. However, after 4 hours of brachial loading, p-γ-H2AX foci were significantly reduced compared to the 4-hour static control group (FIGS. 6C, 6D). This result suggests that brachial loading rapidly disrupts chromatin and reveals sites of DNA DSBs, which are then subsequently repaired by DNA damage proteins such as KU80. To investigate this hypothesis further, a neutral comet assay was performed to assess the total accumulation of DNA DSBs at each time point. The comet assay revealed that DNA damage remained relatively constant after 30 minutes and 4 hours of brachial treatment, compared to the static control. These data suggest that brachial treatment reveals existing DNA DSBs, rather than inducing new DNA damage. Furthermore, the cells were mechanically conditioned for 4 hours, then left at static conditions for an additional 20 hours, following the methods of the typical 7-day mechanical treatment used throughout this study. We found that after 24 hours, the brachial-treated groups demonstrated significantly less DNA damage than the static control (FIGS. 6E, 6F). Altogether, these results suggest that brachial loading rapidly reveals sites of existing DNA damage through p-γ-H2AX foci expression, then enhances the repair of this damage to restore genetic stability. These data suggest that brachial treatment could be revealing existing DNA DSBs, rather than inducing new DNA damage. Furthermore, the cells were also mechanically conditioned for 4 hours, then left in static conditions for an additional 20 hours, following the methods of the typical 7-day mechanical treatment used throughout this study. After 24 hours, the brachial-treated groups demonstrated significantly less DNA damage than the static control. Altogether, these results suggest that brachial loading rapidly reveals sites of existing DNA damage through p-γ-H2AX foci expression, then enhances the repair of this damage to restore genetic stability. A model of a cell being stretched on a flexible membrane (Miles et al., 2023) supports that the loading conditions used would be sufficient to induce opening of chromatin without damage to the DNA.

The nuclear morphology of the mechanically treated MSCs was analyzed with the Nuclear Irregularity Index (NII) plugin in FIJI (Filippi-Chiela et al., 2012). MSCs from a young donor (24 year old female) or an aged donor (81 year old female) were treated with brachial loading or static control treatment for 4 hours. Cells were immunostained for DAPI and processed with the NII plugin to characterize their nuclei as “Normal”, “Apoptotic”, “Senescent”, or “Irregular” (FIG. 14). Results demonstrate that brachial loading causes a significant increase in the frequency of cells with “normal” nuclei, as well as a significant decrease of cells with “irregular” nuclei (FIG. 14B).

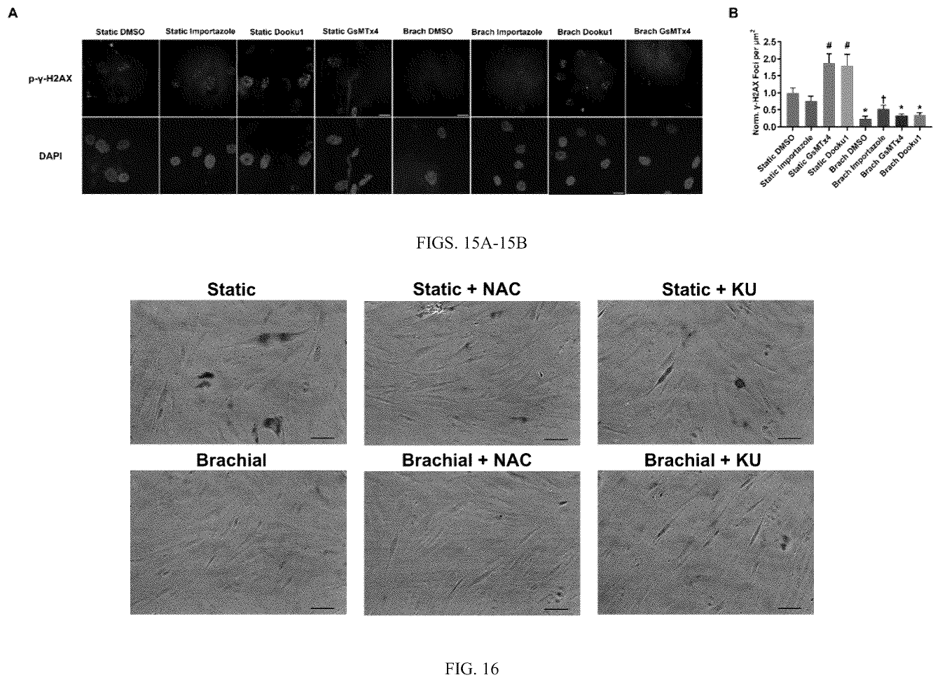

To further investigate the mechanism by which mechanical loading enhances the recognition and repair of DNA damage, the mechanical treatment of aged MSCs was repeated in combination with several pharmaceutical inhibitors of nuclear mechanotransductive signaling. Previous studies have demonstrated that the dynamic tensile loading of MSCs results in altered chromatin condensation that persists long after the completion of mechanical conditioning (Heo et al., 2015). This phenomena, deemed “mechanical memory”, may provide rationale for the long-term functional benefits demonstrated in this study after mechanical conditioning of aged MSCs. Previous work identified that mechanically driven changes in MSC chromatin architecture are dependent on Piezo mechanosensitive calcium channel signaling (Heo et al., 2015). To investigate the role of mechanosensitive signaling in enhanced DNA damage repair, MSCs from an aged donor were treated with brachial loading or static control treatment for 4 hours and co-treated with 40 μM Importazole, a drug that blocks importin-β-dependent nuclear import (Owens et al., 2020), 2 μM Dooku 1, which blocks Yoda1-induced activation of the Piezo1 Ca²⁺ mechanosensitive ion channel (Evans et al., 2018), or GsMTx4, which directly inhibits Piezo1 channel activity (Mousawi et al., 2020). Following the treatments, MSCs were immunostained for p-γ-H2AX foci (FIG. 15). Results demonstrate that enhanced repair of DNA DSB foci is not dependent on mechanosensitive Piezo1 activation (FIG. 15B). Interestingly, co-treatment with brachial loading and Importazole resulted in significantly more p-γ-H2AX foci than treatment with brachial loading alone. This result suggests that while brachial loading is able to mechanically reorganize damaged DNA to enable its repair, this mechanism may be partially dependent on importin-β nuclear transport of proteins such as transcription factors to mediate DNA damage signaling (Cekan et al., 2016; Petrovic & Hoelz, 2022). Furthermore, GsMTx4 and Dooku1 treatment of static control cells caused significantly more p-γ-H2AX foci than static treatment alone, suggesting that Piezo1-mediated nuclear influx of Ca²⁺ may regulate DNA damage response in MSCs (Hamouda et al., 2020).

### Example 7—Inhibition of ATM-Mediated DNA Damage Repair and Oxidative Stress Signaling Reduces Mechanical Protection from Senescence and Proliferation Enhancement

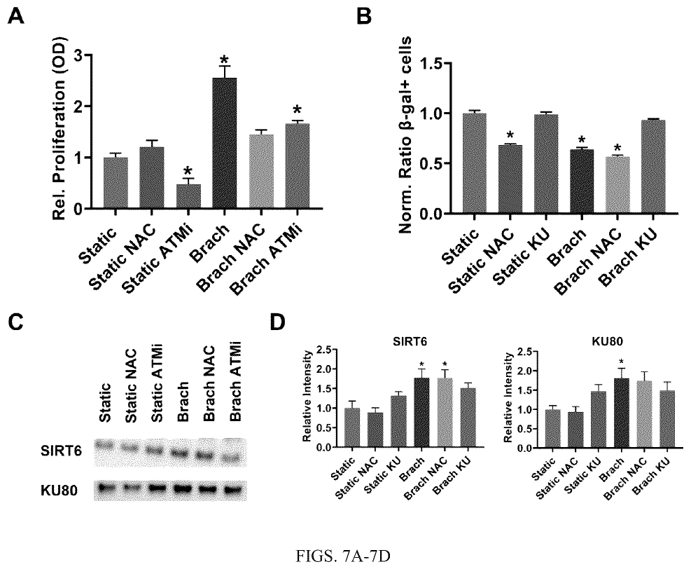

Next, the role of oxidative stress signaling and ATM-mediated DNA damage repair in mechanically rejuvenating the proliferative function of aged MSCs, and protecting them from senescence, was evaluated. MSCs were treated with 5 μM KU55933 (an ATM kinase inhibitor, ATMi) (Wu et al., 2017), 5 mM N-acetyl-L cysteine (NAC), which has been shown to have strong antioxidant effects in MSCs (Watanabe et al., 2018), or control treatments. Cells were then mechanically conditioned and evaluated for DNA synthesis with a BrdU assay. Results showed significantly higher DNA synthesis under brachial loading and brachial+KU55933 treatment, but not under brachial+NAC treatment (FIG. 7A). These results suggest that brachial loading may rely on oxidative stress signaling and the amplification of antioxidant machinery to enhance the proliferative function of the cells. Next, the mechanical and pharmaceutical treatments were repeated and staining for β-galactosidase, a common biomarker of senescent cell phenotypes, performed (Brandl et al., 2011; Xu et al., 2020; Pollock et al., 2015). Results showed a significant reduction in the percentage of β-galactosidase⁺ cells in NAC-treated groups and brachial-treated groups, but not KU55933-treated groups (FIG. 7B, FIG. 16). These results demonstrate the potential of mechanical conditioning to either protect the cells from advancing senescence, or rejuvenate senescent cells into a healthy phenotype, generating a lower population of senescent cell phenotypes following the treatment. These data also suggest that the mechanical enhancement of ATM-mediated DNA damage repair signaling is critical for protecting aged MSCs from the progression of senescence.

Protein expression in pathways related to oxidative stress and DNA damage repair was also evaluated by Western blotting (FIGS. 7C, 7D). A Ponceau stain was run to confirm equal loading of cell lysate protein (FIG. 13C). Results showed significant upregulation of SIRT6 expression with brachial and brachial+NAC treatment, but no significant change with KU55933 treatment. Furthermore, DNA damage repair protein KU80 was upregulated with brachial treatment alone but remained statistically unchanged with brachial+NAC and brachial+KU55933 treatment compared to control groups. Therefore, key regulators of DNA damage repair such as Sirt6 and KU80 rely on ATM signaling that is activated by the mechanical treatment to protect the aged MSCs from senescence. Furthermore, mechanical enhancement of proliferative function is independent of ATM-mediated DNA damage repair signaling and instead relies on the alteration of oxidative stress signaling. Overall, these results lead to the hypothesis that mechanical conditioning activates oxidative stress signaling through SIRT1 and antioxidant proteins such as SOD1, Fox01, and Fox03a to rejuvenate proliferative function in aged MSCs. Mechanical conditioning also activates DNA damage signaling through ATM, SIRT6, and KU80 to rapidly enhance DNA damage repair and protect the aged MSCs from the progression of senescence (FIG. 8).

### Example 8—Mechanical Conditioning Reduces Senescence Related Gene Expression in MSCs from Aged Donors

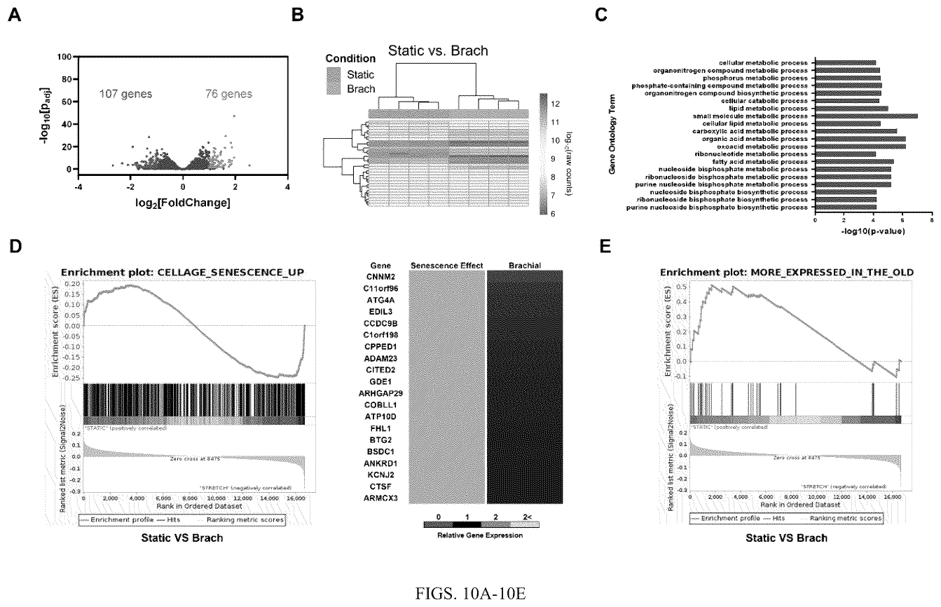

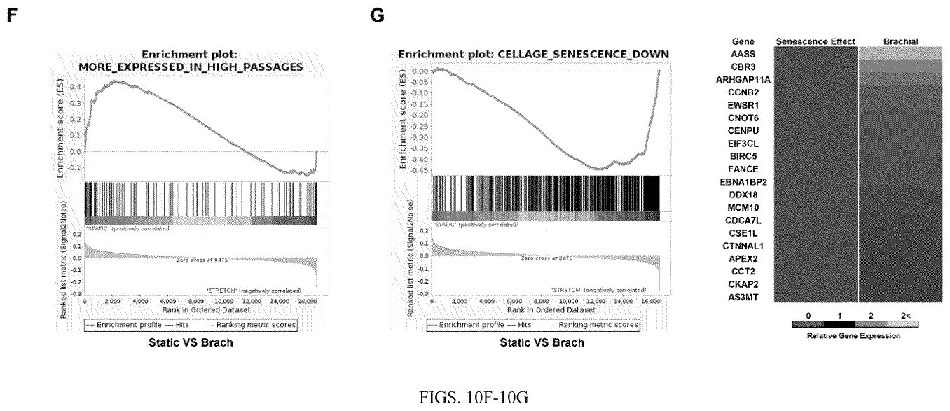

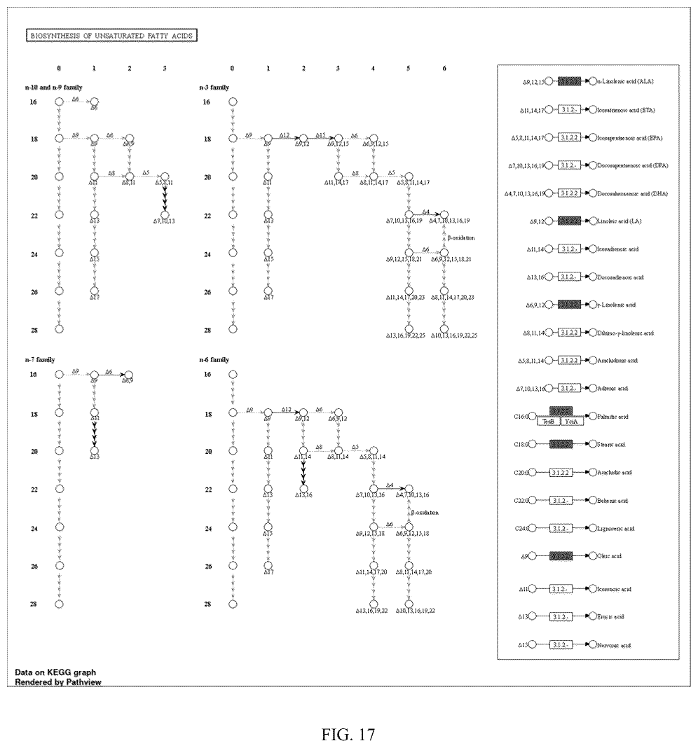

To investigate the effects of mechanical conditioning on gene expression patterns that are correlated with senescence in MSCs, MSCs were obtained from one aged donor (81 years old) and conditioned with brachial loading or static control treatment. Following the treatments, RNA-seq analysis was performed on the cells. A differential gene expression analysis revealed that brachial mechanical loading treatment significantly regulated 183 genes in comparison to the static control treatment (FIG. 10A). Cells treated with brachial loading had similar patterns of gene expression, while MSCs treated with the static control had similar patterns of gene expression (FIG. 10B). Gene ontology analysis of the differential gene expression revealed significant expression increases in gene sets related to cellular metabolic processes and nucleoside biosynthetic processes (FIG. 10C). Previous studies have demonstrated that gene expression related to metabolic processes and protein/nucleotide biosynthetic processes are markedly downregulated in BM-MSCs from aged donors, supporting the critical role of metabolism in regulating stem cell senescence (Sun et al., 2022). KEGG pathway analysis further revealed a significant upregulation of biosynthesis of unsaturated fatty acids pathways in the brachial-treated group, which has been previously reported to be enriched in young, healthy MSCs (FIG. 17) (Yu et al., 2022).

Next, a gene set enrichment analysis (GSEA) was performed on the RNA-seq data. The enrichment scores were calculated comparing the genes expressed between mechanically conditioned cells and control cells for gene sets upregulated in cell senescence (CellAge) (Avelar et al., 2020), upregulated in upregulated in elderly donor derived MSCs (E-MTAB-4879) (Peffers et al., 2016), or and upregulated in high passage MSCs (GSE137186) (Salerno et al., 2020). In all three senescence or aging-related gene sets, a significant reduction in gene enrichment was observed with brachial mechanical loading (FIGS. 10D-10F). Enrichment scores were also calculated for a gene set downregulated in cell senescence (CellAge) (Avelar et al., 2020; Chatsirisupachai et al., 2019), observing a markedly significant increase in gene enrichment with brachial loading (FIG. 10G). Therefore, the mechanical treatment may work to rejuvenate aged MSCs through both the suppression of age-related gene sets, as well as the enhancement of “anti-aging” gene sets, causing a broad reversal of gene expression patterns that are correlated with senescence.

### Example 9—Mechanical Conditioning Modifies Chromatin Accessibility Profile and Causes Enrichment of Transcription Factor Motif Binding in Aged MSCs

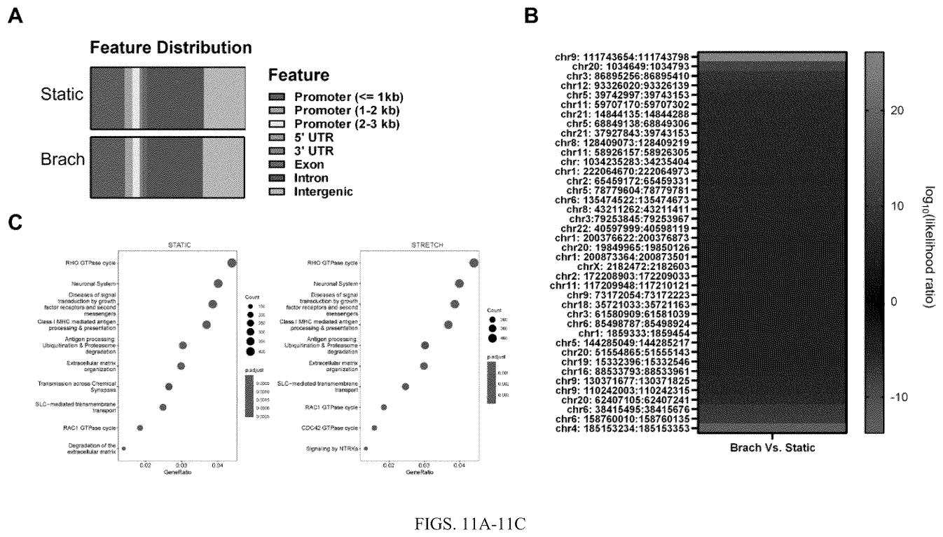

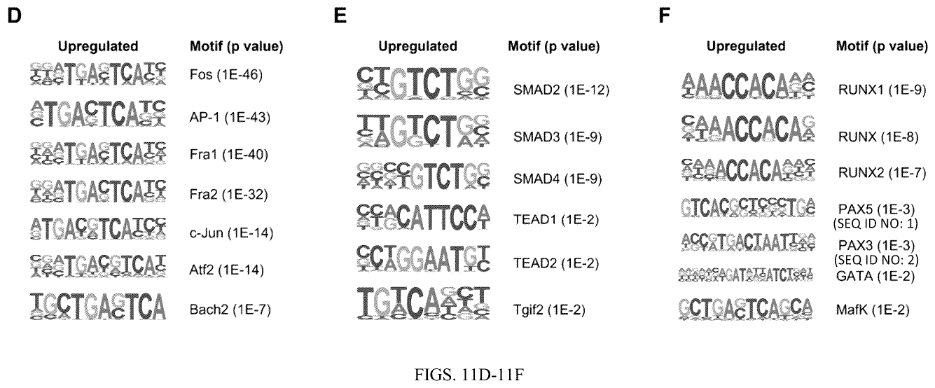

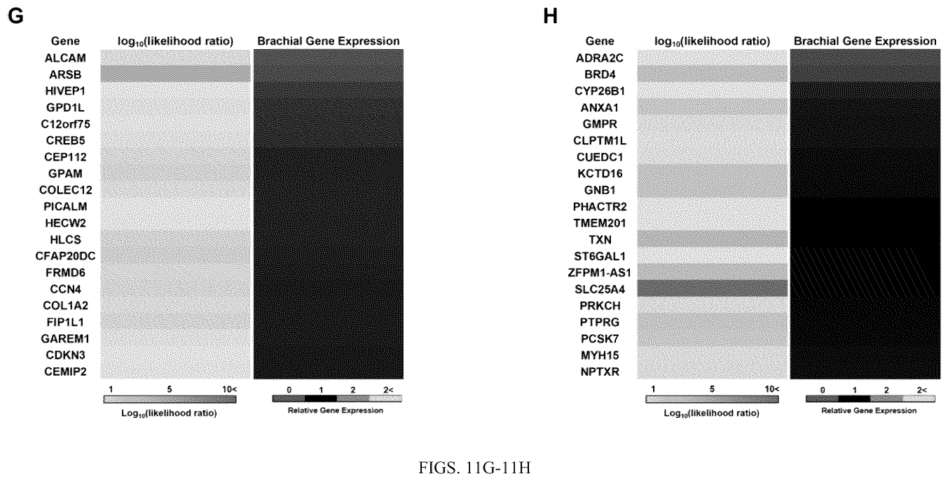

To investigate the mechanical loading effect on altered chromatin accessibility, MSCs were obtained from one aged donor (81 years old) and conditioned with brachial loading or static control treatment. Following the treatments, an assay for transposase-accessible chromatin using sequencing (ATAC-seq) was performed on the cells. The enrichment of accessible chromatin at genomic features between static and brachial treated cells was compared (FIG. 11A). A differential analysis of accessible chromatin peaks revealed 50 genomic regions that were significantly more accessible in static treated cells, and 293 genomic regions significantly more accessible in brachial treated cells. The top 20 most differentially upregulated and downregulated ATAC-seq peaks for brachial vs. static treated cells are listed in FIG. 11B. A Reactome pathway enrichment analysis of biological functions most associated with highly accessible chromatin regions revealed upregulated MAPK family signaling and antigen processing functions with brachial loading treatment (FIG. 11C). Next the Hypergeometric Optimization of Motif EnRichment (HOMER) tool was used to identify transcription factor motif enrichment of accessible chromatin peaks in brachial treated cells (FIGS. 11D-11F) (Heinz et al., 2010). The enriched transcription factor motifs were classified based on their biological function and interaction with other regulatory factors. Several related transcriptional regulators involving the AP-1 complex were identified (FIG. 11D) (Hotfilder et al., 2018). Factors from this list, including c-Jun and AP-1 have been shown to act as co-activators of DNA methyltransferase 1 (DNMT1) to inhibit senescence progression and increase differentiation potential in MSCs (Lin et al., 2014). Transcription factor motifs involved in mechanosignaling pathways, including members of the SMAD and TEAD family, whose enrichment was upregulated with brachial loading were also identified (FIG. 11E). Notably, several of these factors were identified in a previous study as potential drivers of mechanical conditioning-induced signaling to improve MSC proliferation and vascular regeneration (Lee et al., 2021). These findings are consistent with the mechanism identified in this previous study. Several transcription factor motif families, including RUNX, PAX, and GATA, that are known to regulate resident stem cell development, proliferation, and differentiation potential in skeletal muscle and cardiac tissue were also observed (FIG. 11F) (Fitch et al., 2020). Lastly, the ATAC-seq results were integrated with the RNA-seq data to identify potential relationships between chromatin accessibility and differential gene expression (FIG. 11G, 11H). Transcription start sites were mapped to the top 20 differentially enriched peaks for (FIG. 11G) brachial treated cells and (FIG. 11H) static control treated cells, then compared the differential expression of these genes for brachial vs. static treated groups. Upregulated genes include ALCAM, a known marker of normal MSC phenotype, and GPAM, a regulator of healthy mitochondrial metabolism in MSCs (Jiang et al., 2020). Downregulated genes include BRD4, a transcriptional co-activator that activates oncogene-induced senescence and SASP signaling (Tasdemir et al., 2016).

### Example 10—Materials and Methods

Cell lines and cell culture. Human mesenchymal stem cells (Promocell) were cultured in low glucose DMEM medium supplemented with 15% fetal bovine serum, L-glutamine and penicillin/streptomycin. Following trypsinization, cells were seeded on the membranes at 20,000 cells per cm² before mechanical loading. The cells were used between passages 4-6, unless otherwise specified. The MSCs were derived from a donor 1 (Caucasian female, 81 years old), unless otherwise specified. A subset of the studies were performed using MSCs from donor 2 (Caucasian male, 63 years old), or donor 3 (Caucasian male, 68 years old), or donor 4 (Caucasian female, 92 years old). A subset of the studies used MSCs derived from a young, healthy donor (Caucasian female, 24 years old). Human umbilical vein endothelial cells (HUVECs; PromoCell GmbH) were cultured in MCDB 131 medium supplemented with 7.5% fetal bovine serum, L-glutamine and penicillin/streptomycin and EGM-2 SingleQuot Kit (Thermo Fisher Scientific, Inc.). All cells were cultured in an incubator at 37° C. under a 5% CO₂ atmosphere.

Application of biomechanical treatment. Mechanical strain was applied to cell culture using a high throughput system described previously (Lee et al., 2013; Lee & Baker, 2015; Lee et al., 2020). Briefly, cells are cultured on custom made plates that are mounted on a system that applies strain. The cell culture plates were comprised of silicone membranes (0.005″ thickness; Specialty Manufacturing, Inc.) that are sandwiched between two plates, with silicone rubber gaskets at the interfaces to prevent leaking. These cell culture membranes are UV sterilized and coated with 50 μg/mL fibronectin overnight at 37° C. to allow cell adhesion. After cell attachment, the plates are mounted on to the top plate of the system using screws. To apply mechanical strain, a platen with 36 Teflon pistons is moved into the cell culture membrane. The motion is driven by a hygienically sealed, voice coil-type linear motor (Copley Controls). The platen is stabilized using six motion rails mounted with linear motion bearings. The hygienically sealed motor housing has chilled water running through in order to prevent overheating during operation. Unless otherwise specified, loading was applied for 4 hours per day for 7 days with or without 1 μm the EGFR/ErbB-2/ErbB-4 Inhibitor (CAS 881001-19-0; Table 3).

BrdU Proliferation Assay. Following the mechanical and chemical treatments, cells were removed from the stretch plates using 0.05% Trypsin-EDTA, seeded onto optical-bottom 96 well plates at a density of 10,000 cells/well, and allowed to grow for 24 hours. Bromodeoxyuridine (5-bromo-2′-deoxyuridine; BrdU) labeling solution was added to culture media. Cells were washed and stained with the detection and HRP antibodies, following the BrdU assay protocol according to the manufacturer's instructions (Cell Signaling Technology). Absorbance was read at 450 nm using a FlexStation-3 plate reader (Molecular Devices).

Long-term culture expansion assay. Following the mechanical and chemical treatments, cells were removed from the stretch plates using 0.05% Trypsin-EDTA and counted with a hemacytometer. Cells were seeded into standard 6 well culture plates (Corning) at a density of 100,000 cells/well, with an n=2 for each treatment group. Every 7 days, cells were trypsinized, counted, and reseeded into new 6 well plates at an equal cell seeding density. At the end of the study, cumulative doubling events across all passages were calculated for each treatment group.

Cell lysis and immunoblotting. Following the treatments, the cells were lysed in 20 mM Tris with 150 mM NaCl, 1% Triton X-100, 0.1% SDS, 2 mM sodium orthovandate, 2 mM PMSF, 50 mM NaF and a protease inhibitor cocktail (Roche, Inc.). The proteins were separated on a NuPAGE 10% bis-tris midi gel (Novex) and transferred to nitrocellulose membrane using iBlot transfer stack (Novex). The membranes were blocked for one hour in 5% non-fat milk in PBS with 0.01% tween-20 (PBST). After washing twice in PBST, cells were incubated with primary antibodies (Table 1) overnight in 1% non-fat milk at 4° C. The membranes were washed with PBST and incubated at room temperature for two hours with secondary antibody. The membrane was treated with chemiluminescent substrate (SuperSignal West Femto; Thermo Fisher Scientific, Inc.) then imaged using a digital imaging system (Cell Biosciences, Inc.).

Human mesenchymal stem cell multipotency analysis. MSCs at passage 4 were cultured and treated with biomechanical conditioning as described previously. Following the treatments, MSCs removed from the stretch plates with 0.05% Trypsin-EDTA and seeded onto standard culture plates, then expanded under normal culture conditions until passage 8. Following the protocol from the hMSC functional identification kit (R&D Systems; #SC006), P8 MSCs were removed from the culture plates with 0.05% Trypsin-EDTA and seeded onto glass coverslips in 24 well plates at a density of 2.1×10⁴ cells/cm² for the adipogenic differentiation and 4.2×10³ cells/cm² for the osteogenic differentiation. Cells were treated with the respectively provided differentiation media every 3 days for a total of 21 days. Following the differentiation, the cells were fixed in 4% paraformaldehyde in PBS for 10 minutes followed by washing with PBS. Next, samples were blocked and permeabilized with PBS containing 5% FBS, 1% BSA, and 0.3% Triton X-100 PBS for 40 minutes. After washing, cells were incubated with primary antibodies (see Table 2 for specific antibodies and concentrations) in PBS with 1% BSA overnight at 4° C. The samples were then washed twice in PBS with 1% BSA and incubated with secondary antibodies in PBS with 1% BSA for 2 hours in a light protected environment. Cells treated with extensive washes with PBS with 1% BSA prior to flipping the coverslip and mounting on glass slides in anti-fade media (Vector Laboratories, Inc.). The samples were then imaged using confocal fluorescence microscopy (LSM 710 Confocal; Carl Zeiss, Inc.).

Reactive oxygen species assay. MSCs were treated with brachial mechanical loading for 2 hours. Next, culture media was removed, and chemical treatments were added to fresh cell media. Cells received hydrogen peroxide (H2O2; 0.4 mM in PBS), N-acetyl-cysteine (NAC; 5 mM in culture media), or fresh culture media only. The mechanical loading was resumed for 1 additional hour, and chemical treatments were replaced with fresh culture media. The mechanical loading was then resumed for an additional 30 minutes. 4 μL of CellRox DeepRed reagent (ThermoFisher Scientific) was added directly to the cell culture media in each well, and the mechanical loading was resumed for an additional 30 minutes. Following the loading, cells were washed with PBS, removed from the stretch plates using 0.05% Trypsin-EDTA, and centrifuged for 3 minutes at 300G, and supernatant removed. Cells from each treatment group were resuspended in 1 mL room temperature stain buffer (BD Biosciences) in flow cytometry tubes (Falcon). Cell suspensions were centrifuged for 3 minutes at 300G, supernatant removed, and resuspended in 1 mL of stain buffer with 1 μL of Sytox Blue Live/Dead reagent (ThermoFisher Scientific) and incubated for 10 minutes at room temperature in a light-protected environment. Cell suspensions were centrifuged, supernatant removed, and resuspended in 1 mL of 1× fix/perm solution (BD Biosciences) and incubated at 4° C. for 30 minutes. Following fixation, cells were centrifuged with washing buffer two more times, before resuspended in stain buffer and measured. A BD LSR II Fortessa Flow Cytometer (BD Biosciences) was used to measure population fluorescent signals. At least 10,000 events were recorded and further gating and quantification was done through FlowJo software.

Immunocytochemical staining on silicone membranes. Following the treatments, the cells were fixed in 4% paraformaldehyde in PBS for 10 minutes followed by washing and permeabilization with 0.1% Triton X-100 PBS for 5 minutes. Next, samples were blocked with PBS containing 5% FBS and 1% BSA for 40 minutes. After washing, cells were incubated with primary antibodies (see Table 2 for specific antibodies and concentrations) in PBS with 1% BSA overnight at 4° C. The samples were then washed twice in PBS with 1% BSA and incubated with secondary antibodies in PBS with 1% BSA for 2 hours in a light protected environment. Cells treated with extensive washes with PBS with 1% BSA prior to mounting in anti-fade media (Vector Laboratories, Inc.). The samples were then imaged using confocal microscopy with a LSM 710 laser scanning confocal microscope (Carl Zeiss, Inc.).

p-γ-H2AX Foci Analysis. Following confocal imaging, images were analyzed with FIJI software to count p-γ-H2AX histone foci. Three-dimensional image stacks were maximum intensity projected to generate two-dimensional images with 12-bit range of 0-4095. Background signal outside of nuclei boundaries was measured and subtracted from the image. Individual cell nuclei regions of interest were selected, and an intensity threshold of 300 bits was applied. The Analyze Particle plugin was applied to count the p-γ-H2AX foci in each nucleus, with a minimum particle size of 0.5 μm applied. At least 100 individual cell nuclei were analyzed for each treatment group to calculate the mean foci count per cell, mean foci count per nuclear area, and standard errors.

Nuclear Morphometric Analysis. Following confocal imaging, DAPI images were analyzed with the Nuclear Irregularity Index (NII) plugin in FIJI to analyze the nuclear morphology of the cells (Filippi-Chiela et al., 2012). Nuclei were manually marked by defining the nuclei intensity segmentation threshold, as described in the NII plugin manual. Images of nuclei from a young, healthy (24-year-old) MSC line were used to parametrize the “normal nuclei”. Images of nuclei from brachial or static control treated MSCs were included as “treated nuclei”, with at least 100 nuclei analyzed for each group. Nuclei were classified as “Normal”, “Apoptotic”, “Senescent”, or “Irregular” based on measurements of their area, area/box, aspect, radius ratio, and roundness, in comparison to the young “normal nuclei” control.

Comet assay. Cells were treated with brachial mechanical loading at 0.1 Hz and 7.5% maximum strain for 30 minutes, 4 hours, cultured under static conditions, or cultured under static conditions with 0.4 mM H2O2 to induce DNA damage. Following the treatments, a subset of the cells were left in static conditions for 20 hours. Next, a neutral comet assay was performed to quantify the DNA double-strand-breaks, using the Comet Assay Kit (Abcam), and following the manufacturer's protocol. Briefly, the lysis buffer, alkaline solution, and TBE neutral electrophoresis solution were prepared in advance and stored at 4° C. Comet agarose solution was heated at 90° C. for 20 minutes, stirred until homogenized, and pipetted onto the comet slides, then stored horizontally at 4° C. Following the treatments, culture media was removed from the cells, then cells were washed with dPBS and incubated with 25 mM EDTA for 10 minutes at 37° C. Cells were gently removed with pipetting, centrifuged, and resuspended at 2×10⁵ cells/mL in 4° C. dPBS. Cell suspensions were combined with molten comet agarose at a 1/10 (v/v) ratio. 75 μL of cell/agarose suspensions was pipetted into each well of the comet slides, on top of the agarose base layer. Slides were allowed to chill at 4° C. until the agarose solidified. Next, slides were incubated with 4° C. lysis buffer for 60 minutes in light-protected conditions. Lysis buffer was aspirated and replaced with 4° C. alkaline solution and incubated for 30 minutes in light-protected conditions. Alkaline solution was aspirated and replaced with 4° C. TBE electrophoresis solution, then slides were carefully transferred to a horizontal electrophoresis chamber (BioRad). 20 volts were applied for 20 minutes, then slides were transferred to 4° C. deionized water for washing. Lastly, slides were fixed with 4° C. 70% ethanol, then air dried. 1× Vista Green DNA Dye (Abcam) was added to the slides and incubated at room temperature for 15 minutes. The samples were then imaged using confocal microscopy with a LSM 710 laser scanning confocal microscope (Carl Zeiss, Inc.). The OpenComet plugin in FIJI was used to score the comets and quantify the Olive Tail Moment (OTM) for each treatment group (Gyori et al., 2014), with at least 100 comets scored per group.

Conditioning of hMSCs using biochemical factors. For mechanistic experiments involving chemical factors and biological inhibitors, cells were incubated with the chemical treatments as shown in Table 3. The cells were treated with mechanical loading for 4 hours/day under brachial waveform at 0.1 Hz and 7.5% maximum strain or cultured under static conditions. The culture media containing the treatments were replaced on day 3 and day 5 for all 7-day treatments.

β-Galactosidase Analysis. Following the treatments, cell culture media was removed, and cells were washed once with PBS. Cells were fixed and stained for β-Galactosidase following the protocol of the Senescence β-Galactosidase Staining Kit (Cell Signaling Technologies). Following staining, cells were stored with 70% glycerol and imaged using a light microscope (Meiji, Inc.). Images were post-processed in Adobe Photoshop to increase image contrast and visibility of β-Galactosidase+ cells. Total cells and β-Galactosidase+ cells were manually counted in FIJI, with at least 1,000 cells counted per treatment group.

Flow Cytometry. For the characterization of mesenchymal stem cells during long-term culture expansion (passage 5-10), the cells were detached from the culture plate using Accutase (Sigma-Aldrich) and were labeled with fluorescent antibodies according to the R&D Systems human mesenchymal stem cell verification flow kit protocol (R&D FMC020; see Table 4 for the specific antibodies used). Briefly, the detached cells were centrifuged and the supernatant was removed. Fixing and permeabilizing buffer was added while the cells were vortexed and incubated for 40 min. Next, the samples were centrifuged and the supernatant was removed. Cells were then treated with washing buffer containing antibodies for 50 min. Following antibody incubation, cells were centrifuged with washing buffer two more times, then were treated with stain buffer and measured. A BD LSR II Fortessa Flow Cytometer (BD Biosciences) was used to measure population fluorescent signals. At least 10,000 events were recorded and further gating and quantification was done through FlowJo software.

RNA Sequencing and Analysis. Following treatments, RNA was isolated from the cells from four independent wells per group using the Qiagen RNeasy Mini Kit. RNAseq was performed using an Illumina HiSeq 2500 sequencing machine. For sequencing, single reads of 50 base pairs were performed after poly-A mRNA capture using the Poly(A) Tailing Kit (Ambion) and Ultra II Directional RNA Library Prep Kit (NEB) to isolate mRNA and perform dUTP directional preparation of the mRNA library. RNA sequencing was performed by GENEWIZ (Azenta Life Sciences). Gene expression analysis was performed using R. Gene ontology, Pathview, and Kyoto Encyclopedia of Genes and Genomes (KEGG) analysis was performed using the Molecular Signatures Database (En), PANTHER, or ShinyGO (Kanehisa et al., 2021; Luo & Brouwer, 2013; Ge et al., 2020).

Gene set enrichment analysis (GSEA). To define signature gene sets of senescent cells, aged-donor-derived MSCs, or high passage MSCs, we downloaded multiple RNA-seq data sets (Table 5). Obtained RNA-seq data sets were mapped to the human transcripts (GRCh38) with Salmon mapper (v1.1.0) (Patro et al., 2017) and the count of mapped reads was normalized as transcripts per million (TPM) using an R package of tximport (v 1.14.2) (Soneson et al., 2015). Multiple processed RNA-seq data belonging to each cell-type category were considered as replicates and compared to data obtained from mesenchymal stem cells (MSCs) using edgeR (v 3.26.8) (Robinson et al., 2010) to define signature genes for each cell-type. GSEA was performed using FIG. 4 tool (v4.0.3) (Subramanian et al., 2005) with these defined gene sets (p-value 0.001, approximately 500 genes each) and the gene expression data of the brachial treated cells to control static treated cells.

Statistical analysis. All results are shown as mean±standard error of the mean. All experiments used biological replicates that consisted of cells in non-repeated, independent cell culture wells or tissue samples from different animals, unless specified otherwise. Multiple comparisons between groups were analyzed by two-way ANOVA followed by a Tukey post-hoc or a Dunnett post-hoc test when testing multiple comparisons versus a control group. For non-parametric data, multiple comparisons were made using the Kruskal-Wallis test followed by post-hoc testing with the Conover-Iman procedure. A p-value of 0.05 or less was considered statistically significant for all tests.

TABLE 1
Primary Antibodies Used for Immunoblotting

| | | Catalog | Species/ | Dilution |
|---|---|---|---|---|
| Target Protein | Company | # | Isotype | Ratio |
| Cyclin-D1 | Cell Signaling | 2978S | anti-rabbit | 1:500 |
| Phospho-Cyclin-D1 | Cell Signaling | 3300S | anti-rabbit | 1:500 |
| (Thr286) | | | | |
| Akt | Cell Signaling | 4685S | anti-rabbit | 1:500 |
| Phospho-Akt | Cell Signaling | 9271S | anti-rabbit | 1:500 |
| (Ser473) | | | | |
| SIRT1 | Cell Signaling | 9475S | anti-rabbit | 1:500 |
| SIRT3 | Cell Signaling | 2627S | anti-rabbit | 1:500 |
| Phospho-SIRT1 | Cell Signaling | 9787T | anti-rabbit | 1:500 |
| (Ser47) | | | | |
| SIRT3 | Cell Signaling | 2627S | anti-rabbit | 1:500 |
| SIRT2 | Cell Signaling | 9787T | anti-rabbit | 1:500 |
| SIRT6 | Cell Signaling | 12486S | anti-rabbit | 1:500 |
| SIRT7 | Cell Signaling | 9787T | anti-rabbit | 1:500 |
| FOX01 | Cell Signaling | 9946T | anti-rabbit | 1:500 |
| FOX03a | Cell Signaling | 9946T | anti-rabbit | 1:500 |
| Phospho-FOX04 | Cell Signaling | 9946T | anti-rabbit | 1:500 |
| (Thr28) | | | | |
| Phospho-FOX01 | Cell Signaling | 9946T | anti-rabbit | 1:500 |
| (Ser256) | | | | |
| Phospho-FOX03a | Cell Signaling | 9946T | anti-rabbit | 1:500 |
| (Ser253) | | | | |
| SOD1 | Cell Signaling | 2770S | anti-rabbit | 1:500 |
| KU80 | Cell Signaling | 2180S | anti-rabbit | 1:500 |
| ATM | Cell Signaling | 2873S | anti-rabbit | 1:500 |
| Phospho-ATM | Cell Signaling | 5883S | anti-rabbit | 1:500 |
| (Ser1981) | | | | |

TABLE 2
Primary Antibodies Used for Immunostaining

| | | Catalog | Species/ | Dilution |
|---|---|---|---|---|
| Target Protein | Company | # | Isotype | Ratio |
| FABP4 | R&D Systems | SC006 | anti-mouse | 1:500 |
| Osteocalcin | R&D Systems | SC006 | anti-mouse | 1:500 |
| Phospho-γ-H2AX | Cell Signaling | 80312S | anti-mouse | 1:100 |
| (Ser139) | | | | |
| KU80 | Cell Signaling | 2180S | anti-rabbit | 1:400 |

TABLE 3
Reagents for Treating Cells During Mechanistic Studies

| | | Catalog | Concen- |
|---|---|---|---|
| Biomolecule | Company | # | tration |
| EGFR-ErbB2-4 Inhibitor | Sigma-Aldrich | 324840 | 1 μM |
| Hydrogen Peroxide | Sigma-Aldrich | 216763 | 0.4 mM |
| N-Acetyl-L-Cysteine | Sigma-Aldrich | A9165 | 5 mM |
| KU-55933 | Selleck Chemicals | S1092 | 5 μM |
| Importazole | Cell Signaling | 10451S | 40 μM |
| Dooku 1 | Tocris | 6568 | 2 μM |
| GsMTx4 | Tocris | 4912 | 3 μM |

TABLE 4
List of Primary Antibodies Used for Flow Cytometry

| | | Catalog | Fluor. |
|---|---|---|---|
| Target Protein | Company | # | Dye |
| CD90 | R&D Systems | 967542 | APC |
| CD73 | R&D Systems | 967544 | Carboxy- |
| | | | fluorescein |
| CD105 | R&D Systems | 967546 | PerCp/Cy5.5 |
| CD45 (Neg. Cocktail) | R&D Systems | 967548 | PE |
| CD34 (Neg. Cocktail) | R&D Systems | 967548 | PE |
| CD11b (Neg. Cocktail) | R&D Systems | 967548 | PE |
| CD79A (Neg. Cocktail) | R&D Systems | 967548 | PE |
| HLA-DR (Neg. Cocktail) | R&D Systems | 967548 | PE |

TABLE 5
Gene Sets for GSEA Analysis

| Cell Type | GSE ID |
|---|---|
| Human bone-marrow | E-MTAB-4879 |
| mesenchymal stem cells | |
| Human bone-marrow | GSE137186 |
| mesenchymal stem cells | |
| Human mixed cell types | CellAge (HAGR) |
| Human mixed cell types | Gene Expression Signature of |
| | Cellular Senescence (HAGR) |
| Human mixed cell types | M27188 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ahmadi & Rezaie, Ageing and mesenchymal stem cells derived exosomes: Molecular insight and challenges. Cell Biochem Funct 39, 60-66, doi:10.1002/cbf.3602 (2021).
Atashi et al., The Role of Reactive Oxygen Species in Mesenchymal Stem Cell Adipogenic and Osteogenic Differentiation: A Review. Stem Cells and Development 24, 1150-1163, doi:10.1089/scd.2014.0484 (2015).
Avelar, R. A. et al. A multidimensional systems biology analysis of cellular senescence in aging and disease. Genome Biol 21, 91 (2020).
Banimohamad-Shotorbani et al., DNA damage repair response in mesenchymal stromal cells: From cellular senescence and aging to apoptosis and differentiation ability. Ageing Research Reviews 62, 101125, doi: https://doi.org/10.1016/j.arr.2020.101125 (2020).
Benameur et al., Toward an understanding of mechanism of aging-induced oxidative stress in human mesenchymal stem cells. Bio-Medical Materials and Engineering 25, 41-46, doi:10.3233/BME-141247 (2015).
Bonab et al., Aging of mesenchymal stem cell in vitro. BMC Cell Biology 7, 14, doi:10.1186/1471-2121 Jul. 14 (2006).
Brandl et al., Oxidative stress induces senescence in human mesenchymal stem cells. Experimental Cell Research 317, 1541-1547, doi: https://doi.org/10.1016/j.yexcr.2011.02.015 (2011).
Bruder et al., Growth kinetics, self-renewal, and the osteogenic potential of purified human mesenchymal stem cells during extensive subcultivation and following cryopreservation. J Cell Biochem 64, 278-294, doi:10.1002/(sici) 1097-4644 (199702) 64: 2<278::aid-jcb11>3.0.co; 2-f (1997).
Cakouros & Gronthos, Epigenetic Regulation of Bone Marrow Stem Cell Aging: Revealing Epigenetic Signatures associated with Hematopoietic and Mesenchymal Stem Cell Aging. Aging Dis 10, 174-189, doi:10.14336/AD.2017.1213 (2019).
Cekan, P. et al. RCCI-dependent activation of Ran accelerates cell cycle and DNA repair, inhibiting DNA damage-induced cell senescence. Molecular Biology of the Cell 27, 1346-1357 (2016).
Chaib et al., Cellular senescence and senolytics: the path to the clinic. Nature Medicine 28, 1556-1568, doi:10.1038/s41591-022-01923-y (2022).
Chalasani et al., Randomised clinical trial: a leucine-metformin-sildenafil combination (NS-0200) vs placebo in patients with non-alcoholic fatty liver disease. Aliment Pharmacol Ther 47, 1639-1651, doi:10.1111/apt.14674 (2018).
Chatsirisupachai, K., Palmer, D., Ferreira, S. & de Magalhães, J. P. A human tissue-specific transcriptomic analysis reveals a complex relationship between aging, cancer, and cellular senescence. Aging Cell 18, e13041 (2019).
Chen et al., Mechanical stretch induces antioxidant responses and osteogenic differentiation in human mesenchymal stem cells through activation of the AMPK-SIRT1 signaling pathway. Free Radical Biology and Medicine 126, 187-201, doi: https://doi.org/10.1016/j.freeradbiomed.2018.08.001 (2018).
Chen et al., Study on the Dynamic Biological Characteristics of Human Bone Marrow Mesenchymal Stem Cell Senescence. Stem Cells International 2019, 9271595, doi:10.1155/2019/9271595 (2019).
Ciccia & Elledge, The DNA damage response: making it safe to play with knives. Mol Cell 40, 179-204, doi:10.1016/j.molcel.2010.09.019 (2010).
Clayton et al., A comparison of the pro-angiogenic potential of human induced pluripotent stem cell derived endothelial cells and induced endothelial cells in a murine model of peripheral arterial disease. International Journal of Cardiology 234, 81-89, doi: https://doi.org/10.1016/j.ijcard.2017.01.125 (2017).
Denu & Hematti, Effects of Oxidative Stress on Mesenchymal Stem Cell Biology. Oxidative Medicine and Cellular Longevity 2016, 2989076, doi:10.1155/2016/2989076 (2016).
Drela et al., Experimental Strategies of Mesenchymal Stem Cell Propagation: Adverse Events and Potential Risk of Functional Changes. Stem Cells International 2019, 7012692, doi:10.1155/2019/7012692 (2019).
Evans, E. L. et al. Yoda1 analogue (Dooku1) which antagonizes Yoda1-evoked activation of Piezo1 and aortic relaxation. British Journal of Pharmacology 175, 1744-1759 (2018).
Fafián-Labora et al., Effect of aging on behaviour of mesenchymal stem cells. World J Stem Cells 11, 337-346, doi:10.4252/wjsc.v11.16.337 (2019).
Filippi-Chiela, E. C. et al. Nuclear morphometric analysis (NMA): screening of senescence, apoptosis and nuclear irregularities. PLOS One 7, e42522 (2012).
Fitch, S. R. et al. Gata3 targets Runx1 in the embryonic haematopoietic stem cell niche. IUBMB Life 72, 45-52 (2020).
Fridman & Tainsky, Critical pathways in cellular senescence and immortalization revealed by gene expression profiling. Oncogene 27, 5975-5987, doi:10.1038/onc.2008.213 (2008).
Ge, S. X., Jung, D. & Yao, R. ShinyGO: a graphical gene-set enrichment tool for animals and plants. Bioinformatics 36, 2628-2629 (2020).
Gharibi et al., Inhibition of Akt/mTOR Attenuates Age-Related Changes in Mesenchymal Stem Cells. STEM CELLS 32, 2256-2266, doi: https://doi.org/10.1002/stem.1709 (2014).
Gyori, B. M., Venkatachalam, G., Thiagarajan, P. S., Hsu, D. & Clement, M.-V. OpenComet: An automated tool for comet assay image analysis. Redox Biology 2, 457-465 (2014).
Halaby et al., ATM protein kinase mediates full activation of Akt and regulates glucose transporter 4 translocation by insulin in muscle cells. Cellular Signalling 20, 1555-1563, doi: https://doi.org/10.1016/j.cellsig.2008.04.011 (2008).
Hamouda, M. S., Labouesse, C. & Chalut, K. J. Nuclear mechanotransduction in stem cells. Current Opinion in Cell Biology 64, 97-104 (2020).
Heinz, S. et al. Simple combinations of lineage-determining transcription factors prime cis-regulatory elements required for macrophage and B cell identities. Mol Cell 38, 576-589 (2010).
Heo, S.-J. et al. Biophysical Regulation of Chromatin Architecture Instills a Mechanical Memory in Mesenchymal Stem Cells. Scientific Reports 5, 16895 (2015).
Hiramoto et al., Interventions for lower extremity peripheral artery disease. Nat Rev Cardiol 15, 332-350, doi:10.1038/s41569-018-0005-0 (2018).
Hladik et al., Long-term culture of mesenchymal stem cells impairs ATM-dependent recognition of DNA breaks and increases genetic instability. Stem cell research & therapy 10, 218-218, doi:10.1186/s13287-019-1334-6 (2019).
Hotfilder, M., Mallela, N., Seggewiß, J., Dirksen, U. & Korsching, E. Defining a Characteristic Gene Expression Set Responsible for Cancer Stem Cell-Like Features in a Sub-Population of Ewing Sarcoma Cells CADO-ES1. Int J Mol Sci 19 (2018).
Huang et al., Cellular senescence: the good, the bad and the unknown. Nature Reviews Nephrology 18, 611-627, doi:10.1038/s41581-022-00601-z (2022).
Huang et al., Differentiation of Allogeneic Mesenchymal Stem Cells Induces Immunogenicity and Limits Their Long-Term Benefits for Myocardial Repair. Circulation 122, 2419-2429, doi:10.1161/CIRCULATIONAHA.110.955971 (2010).
Isakova et al., Allo-Reactivity of Mesenchymal Stem Cells in Rhesus Macaques Is Dose and Haplotype Dependent and Limits Durable Cell Engraftment In Vivo. PLOS ONE 9, e87238, doi:10.1371/journal.pone.0087238 (2014).
Jiang, D. et al. Bioenergetic Crosstalk between Mesenchymal Stem Cells and various Ocular Cells through the intercellular trafficking of Mitochondria. Theranostics 10, 7260-7272 (2020).
Kanehisa, M., Furumichi, M., Sato, Y., Ishiguro-Watanabe, M. & Tanabe, M. KEGG: integrating viruses and cellular organisms. Nucleic Acids Research 49, D545-D551 (2021).
Khanh et al., Rejuvenation of mesenchymal stem cells by extracellular vesicles inhibits the elevation of reactive oxygen species. Scientific Reports 10, 17315, doi:10.1038/s41598-020-74444-8 (2020).
Ki et al., Global transcriptional profiling reveals distinct functions of thymic stromal subsets and age-related changes during thymic involution. Cell Rep 9, 402-415, doi:10.1016/j.celrep.2014.08.070 (2014).
Kim et al., Age-related alterations in mesenchymal stem cells related to shift in differentiation from osteogenic to adipogenic potential: Implication to age-associated bone diseases and defects. Mechanisms of Ageing and Development 133, 215-225, doi: https://doi.org/10.1016/j.mad.2012.03.014 (2012).
Kohn et al., Cost and inpatient burden of peripheral artery disease: Findings from the National Inpatient Sample. Atherosclerosis 286, 142-146, doi:10.1016/j.atherosclerosis.2019.05.026 (2019).
Kornienko et al., High doses of synthetic antioxidants induce premature senescence in cultivated mesenchymal stem cells. Scientific Reports 9, 1296, doi:10.1038/s41598-018-37972-y (2019).
Kosar et al., Senescence-associated heterochromatin foci are dispensable for cellular senescence, occur in a cell type- and insult-dependent manner and follow expression of p16 (ink4a). Cell Cycle 10, 457-468, doi:10.4161/cc.10.3.14707 (2011).
Kouchakian et al., The Clinical Trials of Mesenchymal Stromal Cells Therapy. Stem Cells International 2021, 1634782, doi:10.1155/2021/1634782 (2021).
Kuang et al., Functional and Molecular Changes of MSCs in Aging. Curr Stem Cell Res Ther 10, 384-391, doi:10.2174/1574888×10666150211162933 (2015).
Lee, J. & Baker, A. B. Computational analysis of fluid flow within a device for applying biaxial strain to cultured cells. J Biomech Eng 137, 051006 (2015).
Lee & Paull, Cellular functions of the protein kinase ATM and their relevance to human disease. Nature reviews. Molecular cell biology 22, 796-814, doi:10.1038/s41580-021-00394-2 (2021b).
Lee, J. et al. A high throughput screening system for studying the effects of applied mechanical forces on reprogramming factor expression. Sci Rep 10, 15469 (2020).
Lee et al., A novel system for studying mechanical strain waveform-dependent responses in vascular smooth muscle cells. Lab Chip 13, 4573-4582, doi:10.1039/c31c50894c (2013).
Lee et al., Antioxidant effects of Cirsium setidens extract on oxidative stress in human mesenchymal stem cells. Mol Med Rep 14, 3777-3784, doi:10.3892/mmr.2016.5706 (2016).
Lee et al., High Throughput Mechanobiological Screens Enable Mechanical Priming of Pluripotency in Mouse Fibroblasts. bioRxiv (2018).
Lee et al., Mechanobiological conditioning of mesenchymal stem cells for enhanced vascular regeneration. Nat Biomed Eng 5, 89-102, doi:10.1038/s41551-020-00674-w (2021a).
Lee et al., Sirtuin signaling in cellular senescence and aging. BMB Rep 52, 24-34, doi:10.5483/BMBRep.2019.52.1.290 (2019).
Levy et al., Shattering barriers toward clinically meaningful MSC therapies. Science Advances 6, caba6884, doi:doi:10.1126/sciadv.aba6884 (2020).
Li et al., How to Improve the Survival of Transplanted Mesenchymal Stem Cell in Ischemic Heart? Stem Cells Int 2016, 9682757, doi:10.1155/2016/9682757 (2016).
Liang & Ghaffari, Stem Cells, Redox Signaling, and Stem Cell Aging. Antioxidants & Redox Signaling 20, 1902-1916, doi:10.1089/ars.2013.5300 (2013).
Lin et al., Bone marrow mesenchymal stem cells: Aging and tissue engineering applications to enhance bone healing. Biomaterials 203, 96-110, doi:10.1016/j.biomaterials.2018.06.026 (2019).
Liu et al., (2020). “Senescence in Mesenchymal Stem Cells: Functional Alterations, Molecular Mechanisms, and Rejuvenation Strategies.” Frontiers in Cell and Developmental Biology 8 (258).
Lin, S. P. et al. RB maintains quiescence and prevents premature senescence through upregulation of DNMT1 in mesenchymal stromal cells. Stem Cell Reports 3, 975-986 (2014).
Loomans et al., Endothelial Progenitor Cell Dysfunction. Diabetes 53, 195, doi:10.2337/diabetes.53.1.195 (2004).
Lunyak et al., Mesenchymal Stem Cells Secretory Responses: Senescence Messaging Secretome and Immunomodulation Perspective. Frontiers in Genetics 8 doi:10.3389/fgene.2017.00220 (2017).
Luo, W. & Brouwer, C. Pathview: an R/Bioconductor package for pathway-based data integration and visualization. Bioinformatics 29, 1830-1831 (2013).
McCauley, B. S. et al. Altered chromatin states drive cryptic transcription in aging mammalian stem cells. Nature Aging 1, 684-697 (2021).
Meng et al., Senescent mesenchymal stem/stromal cells and restoring their cellular functions. World J Stem Cells 12, 966-985, doi:10.4252/wjsc.v12.19.966 (2020).
Miles, M. W., Ashirov, D., Demkov, A., Sices, A. & Baker, A. B. A Computational Model of Mechanical Stretching of Cultured Cells on a Flexible Membrane. Revision Under Review at Biotechnology & Bioengineering (2023).
Mousawi, F. et al. Chemical activation of the Piezo1 channel drives mesenchymal stem cell migration via inducing ATP release and activation of P2 receptor purinergic signaling. Stem Cells 38, 410-421 (2020).
Murphy et al., Mesenchymal stem cells: environmentally responsive therapeutics for regenerative medicine. Exp Mol Med 45, e54, doi:10.1038/emm.2013.94 (2013).
Oliver et al., Differentiation-Related Response to DNA Breaks in Human Mesenchymal Stem Cells. STEM CELLS 31, 800-807, doi: https://doi.org/10.1002/stem.1336 (2013).
Onn et al., SIRT6 is a DNA double-strand break sensor. eLife 9, e51636, doi:10.7554/eLife.51636 (2020).
Ouyang et al., Mechanical stimulation promotes the proliferation and the cartilage phenotype of mesenchymal stem cells and chondrocytes co-cultured in vitro. Biomedicine & Pharmacotherapy 117, 109146, doi: https://doi.org/10.1016/j.biopha.2019.109146 (2019).
Owens, D. J. et al. Lamin Mutations Cause Increased YAP Nuclear Entry in Muscle Stem Cells. Cells 9 (2020).
Ozkul & Galderisi, The Impact of Epigenetics on Mesenchymal Stem Cell Biology. Journal of Cellular Physiology 231, 2393-2401, doi: https://doi.org/10.1002/jcp.25371 (2016).
Park et al., Lactoferrin Protects Human Mesenchymal Stem Cells from Oxidative Stress-Induced Senescence and Apoptosis. J Microbiol Biotechnol 27, 1877-1884, doi:10.4014/jmb.1707.07040 (2017).
Patro, R., Duggal, G., Love, M. I., Irizarry, R. A. & Kingsford, C. Salmon provides fast and bias-aware quantification of transcript expression. Nat Methods 14, 417-419 (2017).
Peach et al., Diagnosis and management of peripheral arterial disease. BMJ: British Medical Journal 345, e5208, doi:10.1136/bmj.e5208 (2012).
Peffers et al., Age-related changes in mesenchymal stem cells identified using a multi-omics approach. Eur Cell Mater 31, 136-159, doi:10.22203/ecm.v031a10 (2016).
Petrovic, S. & Hoelz, A. Forced entry into the nucleus. Nature Cell Biology 24, 810-812 (2022).
Pinto & Flaus, Structure and function of histone H2AX. Subcell Biochem 50, 55-78, doi:10.1007/978-90-481-3471-7_4 (2010).
Pollock et al., Clinical mesenchymal stromal cell products undergo functional changes in response to freezing. Cytotherapy 17, 38-45, doi: https://doi.org/10.1016/j.jcyt.2014.06.008 (2015).
Pyo et al., Mechanisms of Aging and the Preventive Effects of Resveratrol on Age-Related Diseases. Molecules 25 (2020).
Robinson, M. D., McCarthy, D. J. & Smyth, G. K. edgeR: a Bioconductor package for differential expression analysis of digital gene expression data. Bioinformatics 26, 139-140 (2010).
Roy, I. M., Nadar, P. S. & Khurana, S. Neutral Comet Assay to Detect and Quantitate DNA Double-Strand Breaks in Hematopoietic Stem Cells. Bio-protocol 11, e4130 (2021).
Salerno et al., MMP13 and TIMP1 are functional markers for two different potential modes of action by mesenchymal stem/stromal cells when treating osteoarthritis. STEM CELLS 38, 1438-1453, doi: https://doi.org/10.1002/stem.3255 (2020).
Siegel et al., Phenotype, donor age and gender affect function of human bone marrow-derived mesenchymal stromal cells. BMC Medicine 11, 146, doi:10.1186/1741-7015-11-146 (2013).
Soneson, C., Love, M. I. & Robinson, M. D. Differential analyses for RNA-seq: transcript-level estimates improve gene-level inferences. F1000Res 4, 1521 (2015).
Stolzing et al., Age-related changes in human bone marrow-derived mesenchymal stem cells: consequences for cell therapies. Mech Ageing Dev 129, 163-173, doi:10.1016/j.mad.2007.12.002 (2008).
Subramanian, A. et al. Gene set enrichment analysis: a knowledge-based approach for interpreting genome-wide expression profiles. Proc Natl Acad Sci USA 102, 15545-15550 (2005).
Sun, Y. et al. RNA sequencing profiles reveal dynamic signaling and glucose metabolic features during bone marrow mesenchymal stem cell senescence. Cell & Bioscience 12, 62 (2022).
Tang et al., SIRT7-mediated ATM deacetylation is essential for its deactivation and DNA damage repair. Science Advances 5, eaav1118, doi:doi:10.1126/sciadv.aav1118 (2019).
Tasdemir, N. et al. BRD4 Connects Enhancer Remodeling to Senescence Immune Surveillance. Cancer Discovery 6, 612-629 (2016).
Turinetto et al., Senescence in Human Mesenchymal Stem Cells: Functional Changes and Implications in Stem Cell-Based Therapy. International Journal of Molecular Sciences 17, doi:10.3390/ijms17071164 (2016).
Vecellio et al., The Histone Acetylase Activator Pentadecylidenemalonate 1b Rescues Proliferation and Differentiation in the Human Cardiac Mesenchymal Cells of Type 2 Diabetic Patients. Diabetes 63, 2132, doi:10.2337/db 13-0731 (2014).
Vono et al., Oxidative Stress in Mesenchymal Stem Cell Senescence: Regulation by Coding and Noncoding RNAs. Antioxidants & Redox Signaling 29, 864-879, doi:10.1089/ars.2017.7294 (2017).
Watanabe et al., Preconditioning of bone marrow-derived mesenchymal stem cells with N-acetyl-L-cysteine enhances bone regeneration via reinforced resistance to oxidative stress. Biomaterials 185, 25-38, doi: https://doi.org/10.1016/j.biomaterials.2018.08.055 (2018).
Wu et al., Early Passage Mesenchymal Stem Cells Display Decreased Radiosensitivity and Increased DNA Repair Activity. Stem Cells Transl Med 6, 1504-1514, doi:10.1002/sctm. 15-0394 (2017).
Xu et al., Ganoderic Acid D Protects Human Amniotic Mesenchymal Stem Cells against Oxidative Stress-Induced Senescence through the PERK/NRF2 Signaling Pathway. Oxidative Medicine and Cellular Longevity 2020, 8291413, doi:10.1155/2020/8291413 (2020).
Yong et al., Mesenchymal Stem Cell Therapy for Ischemic Tissues. Stem Cells International 2018, 8179075, doi:10.1155/2018/8179075 (2018).
Yu et al., The replicative senescent mesenchymal stem/stromal cells defect in DNA damage response and anti-oxidative capacity. Int J Med Sci 15, 771-781, doi:10.7150/ijms.24635 (2018).
Yu, X. et al. A comprehensive analysis of age-related metabolomics and transcriptomics reveals metabolic alterations in rat bone marrow mesenchymal stem cells. Aging (Albany NY) 14, 1014-1032 (2022).
Yuan et al., NAD+/NADH redox alterations reconfigure metabolism and rejuvenate senescent human mesenchymal stem cells in vitro. Communications Biology 3, 774, doi:10.1038/s42003-020-01514-y (2020).
Zaim et al., Donor age and long-term culture affect differentiation and proliferation of human bone marrow mesenchymal stem cells. Annals of Hematology 91, 1175-1186, doi:10.1007/s00277-012-1438-x (2012).
Zhang et al., Mice deficient in both Mn superoxide dismutase and glutathione peroxidase-1 have increased oxidative damage and a greater incidence of pathology but no reduction in longevity. J Gerontol A Biol Sci Med Sci 64, 1212-1220, doi:10.1093/gerona/glp132 (2009).
Zhang et al., Sirtuin3 protects aged human mesenchymal stem cells against oxidative stress and enhances efficacy of cell therapy for ischaemic heart diseases. J Cell Mol Med 22, 5504-5517, doi:10.1111/jcmm. 13821 (2018).
Zhou et al., Mesenchymal Stem Cell Senescence and Rejuvenation: Current Status and Challenges. Frontiers in Cell and Developmental Biology 8, doi:10.3389/fcell.2020.00364 (2020).
Zhu, G., Chai, J., Ma, L., Duan, H. & Zhang, H. Downregulated microRNA-32 expression induced by high glucose inhibits cell cycle progression via PTEN upregulation and Akt inactivation in bone marrow-derived mesenchymal stem cells. Biochemical and Biophysical Research Communications 433, 526-531 (2013).

1. A method of preparing a therapeutic cell population, the method comprising:
(a) adhering a starting population of cells on a flexible or expandable surface;
(b) applying a brachial waveform of mechanical stretch to the starting population of cells, thereby generating a second population of cells;
(c) expanding the second population of cells under static conditions; and
(d) applying a brachial waveform of mechanical stretch to the expanded second population of cells in the presence of at least one pharmacological agent that induces differentiation of the cells, thereby generating the therapeutic population of cells.

2. The method of claim 1, wherein the brachial waveform mechanical stretch in step (b) and/or step (d) is applied to the cells for 1 day, 2 days, 3 days, 4 days, 5 days, 6 days, 7 days, 8 days, 9 days, or 10 days.

3. The method of claim 1, wherein the brachial waveform mechanical stretch in step (b) and/or step (d) is applied to the cells for about 1 hour, about 2 hours, about 3 hours, about 4 hours, about 5 hours, about 6 hours, about 7 hours, or about 8 hours per day.

4. The method of claim 1, wherein the brachial waveform mechanical stretch in step (b) and/or step (d) is applied to the cells for about 4 hours per day for 7 days.

5. The method of claim 1, wherein the brachial waveform in step (b) and/or step (d) has a frequency of 0.01 Hz-1.00 Hz.

6. (canceled)

7. The method of claim 1, wherein the brachial waveform in step (b) and/or step (d) has a magnitude of strain of 0.1% to 17.5%.

8. (canceled)

9. The method of claim 1, wherein the brachial waveform in step (b) and/or step (d) has a magnitude of 7.5% strain and a frequency of 0.1 Hz.

10. The method of claim 1, wherein step (b) comprises applying a brachial waveform of mechanical stretch to the starting population of cells for about 4 hours per day for about 7 days; step (c) comprises expanding the second population of cells under static conditions for at least about 1 week, 2 weeks, 3 weeks, 4 weeks, 5 weeks, 6 weeks, 7 weeks, or 8 weeks; and step (d) comprises applying a brachial waveform of mechanical stretch to the expanded second population of cells in the presence of at least one pharmacological agent that induces differentiation of the cells for about 4 hours per day for about 7 days.

11. The method of claim 1, wherein the at least one pharmacological agent that induces differentiation of the cells is an agent that inhibits signaling of at least one ErbB family protein.

12. The method of claim 1, wherein step (b) comprises applying a brachial waveform of mechanical stretch to the starting population of cells for about 4 hours per day for about 7 days; step (c) comprises expanding the second population of cells under static conditions for at least about 1 week, 2 weeks, 3 weeks, 4 weeks, 5 weeks, 6 weeks, 7 weeks, or 8 weeks; and step (d) comprises applying a brachial waveform of mechanical stretch to the expanded second population of cells in the presence of at least one pharmacological agent that inhibits signaling of at least one ErbB family protein for about 4 hours per day for about 7 days.

13. The method of claim 11, wherein the agent that inhibits signaling of at least one ErbB family protein inhibits EGFR/ErbB1 signaling, HER2/ErbB2 signaling, HER4/ErbB4 signaling, or EGFR/PKC signaling.

14. (canceled)

15. The method of claim 11, wherein the agent that inhibits signaling of at least one ErbB family protein is a kinase inhibitor.

16. The method of claim 15, wherein the kinase inhibitor is an EGFR/Erb-2/4 inhibitor or a PKCβII/EGFR inhibitor.

17-25. (canceled)

26. The method of claim 1, wherein the starting population of cells is senescent.

27-43. (canceled)

44. The method of claim 1, further comprising
(e) encapsulating the population of therapeutic cells in a gel delivery vehicle.

45. The method of claim 44, wherein the gel delivery vehicle is an alginate-RGD-collagen gel delivery vehicle.

46. The method of claim 1, further comprising
(e) culturing the population of therapeutic cells on a tissue engineering scaffold to generate a tissue-engineered construct.

47. A population of therapeutic cells produced by the method of claim 1.

48. A composition comprising the population of therapeutic cells of claim 47.

49-52. (canceled)

53. A method of treating a patient in need thereof comprising administering a therapeutically effective amount of the composition of claim 48 to the patient.

54-63. (canceled)


SEQUENCE LISTING (2 sequences)

SEQ ID NO: 1 - DNA, 14 bp, synthetic construct
  source 1..14: mol_type = other DNA
gtcayrshnnntgv

SEQ ID NO: 2 - DNA, 15 bp, synthetic construct
  source 1..15: mol_type = other DNA
nsyrtrdmhrawnnn